(19) United States
(12) Patent Application Publication
(10) Pub. No.: US 2009/0215092 A1
(43) Pub. Date: Aug. 27, 2009

(54) Antibodies for Anthrax

(75) Inventors: Tracey Elizabeth Love, Salisbury (GB); Caroline Redmond, Salisbury (GB); Carl Nicholas Mayers, Salisbury (GB)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: THE SECRETARY OF STATE FOR DEFENCE, Salisbury, Wiltshire (GB)

(21) Appl. No.: 11/988,760

(22) PCT Filed: Jul. 12, 2006
(86) PCT No.: PCT/GB2006/002560

§ 371 Date: Jan. 14, 2008

(30) Foreign Application Priority Data
Jul. 13, 2005 (GB) 0514319.3

(51) Int. Cl. G01N 33/569; C07K 16/18; C12N 15/11; C40B 30/04

(52) U.S. Cl. 435/7.32; 530/387.1; 536/23.53; 530/387.2; 506/9

(57) ABSTRACT

A targeted approach is described for the production of biological recognition elements capable of fast, specific detection of anthrax spores on biosensor surfaces. Single chain antibodies (scFvs) are produced to EA1, a Bacillus anthracis S-layer protein that is also present, although is not identical, in related Bacillus species. These antibodies detect Bacillus anthracis EA1 protein and intact spores with a high degree of specificity, but do not detect other Bacillus species. Recombinant anti-EA1 scFvs were isolated from an B. anthracis immune library that contained antibody genes raised against B. anthracis spores and purified exosporium. Two approaches for scFv selection are disclosed; standard (non-competitive) panning, and competitive panning. The non-competitive bio-panning strategy isolated scFvs that recognised EA1 from B. anthracis, but also cross-reacted with other Bacillus species. In contrast, the competitive panning approach used S-layer proteins from other Bacillus species to compete out any cross reacting antibodies, generating scFvs that were highly specific to B. anthracis EA1 and demonstrated apparent nanomolar binding affinities. The specific, real time detection of B. anthracis spores was demonstrated with these scFvs by using an evanescent wave biosensor, the Resonant Mirror. The approach described here can be used to generate specific antibodies to any desired target where homologous proteins also exist in closely related species, and demonstrates clear advantages to using recombinant technology to produce biological recognition elements for detection of biological threat agents.

The current invention is concerned with antibodies for the species Bacillus anthracis and uses thereof.

Throughout the following specification various references are made to scientific publications, the contents of which are incorporated herein by reference. For convenience, these publications are listed at the end of the description of the invention and are referred to throughout the text by their Reference number.

The first step in developing an immunoassay for the detection of a pathogen is usually the development of a highly specific antibody that will recognise the live pathogen. These antibodies can be either poly- or monoclonal, and are usually produced against the whole organism. Although often successful, it can prove difficult to obtain antibodies with sufficient specificity by this approach. In the case of polyclonal antibodies raised against whole cells or spores, the antibody will often recognise a range of undefined antigens and epitopes. Furthermore, these epitopes may be conserved between closely related, non-target species. Obtaining a specific antibody may also be difficult if unique targets are rare or are not immunodominant. In the case of a monoclonal antibody only one epitope, often of an unknown protein, is recognised. Monoclonal antibodies, although directed against a single epitope, may still not be able to discriminate between highly conserved epitopes in closely related species. The use of antibodies against unidentified and uncharacterised proteins or epitopes gives significant limitations to the sensitivity and specificity of a detection assay, as well as reducing confidence in the results obtained.

In addition to the problems observed with lack of specificity or the recognition of conserved target proteins, there are other drawbacks to producing antibodies by traditional methods. Antibody production is reliant on the mammalian immune response, making it difficult to produce antibodies to toxic substances, rare epitopes, or non-immunodominant epitopes that could be suitable targets for detection assays (Reference 3). In the case of polyclonal antibodies, a specific target can be identified and specific antibodies purified through the use of affinity purification (Reference 22). This requires considerable knowledge of suitable targets and requires a large amount of purified target, often a recombinant protein. It is an expensive and time consuming procedure, particularly for large scale production.

Production of antibodies by traditional methods has provided a platform that is the basis of many detection and diagnostic technologies. However, production of these natural molecules has often required the modification of detection technologies to optimise performance. For example, this may involve the use of additional reagents (Reference 4), optimisation of assay conditions (Reference 27), development or modification of new or existing technologies Reference 21). Advances in recombinant DNA technology and computational molecular biology have allowed the production of alternatives to traditional antibodies that can be modified to suit the requirements of the detector and assay design (References 16, 9, 23 and 11). One of the distinct advantages of recombinant antibodies is that they can be designed or selected to discriminate between very similar proteins. This can be done either by experimental methods or by a process of rational design (References 5, 19, 17, 2, 12 and 26). The use of a carefully selected target antigen that contains highly specific, epitopes allows an increase in the specificity, sensitivity and confidence of a detection assay (Reference 15). An objective of the current invention is the production of highly specific single chain antibodies to Bacillus anthracis. Antibodies to the highly antigenic Bacillus spores have been produced with a high degree of success; making antibodies that are specific solely to Bacillus anthracis and no other Bacillus species is much more challenging.

Genetic analysis has revealed that Bacillus anthracis is very closely related to Bacillus cereus and Bacillus thuringiensis (Reference 24). Helgason et al. (Reference 6) have suggested that all three could be regarded as the same species and that B. anthracis and B. thuringiensis evolved from a common ancestral species (B. cereus) through the acquisition of plasmids encoding toxin genes, such as pXO1 in the case of B. anthracis. Many of the proteins that have been identified for B. anthracis spores also have homologues within other members of the B. cereus group (References 8, 28 and 25). Completely unique targets for detection and diagnosis may be very rare and/or of low abundance, making identification of these proteins and production of antibodies by traditional methods difficult. An approach that would allow for the production of antibodies that can discriminate between closely related species is vital for sensitive and specific detection of B. anthracis spores. Zhou et al (Reference 31) reported an approach to obtain antibodies to B. anthracis spores from a naïve human scFv library, but cross reactivity with closely related Bacillus species was observed. Williams et al. (Reference 29) report the development of peptide ligands to B. anthracis spores, but the ligand target was unknown. Both of these approaches used whole spores where the detection target was not defined—the current inventors have used a targeted approach to ligand production. Single chain antibodies have been produced that can specifically detect B. anthracis spores through recognition of an individual, characterised protein target.

The protein target we selected from B. anthracis is the surface layer protein EA1. EA1 is a vegetative cell protein; however, results from other work suggest that it is also present in spore preparations (References 25 and 30). It has been suggested that EA1 was a contaminant within spore preparations and could be removed through the use of Urografin purification (Reference 30). The current inventors have used EA1 as a model system to demonstrate means of rapidly selecting for antibodies to an individual, non-specific, protein target. EA1 is known to have homologues in other Bacillus species. The inventors have selected for recognition elements specific to B. anthracis EA1 that could demonstrate specific detection of B. anthracis spores without cross reactivity with other Bacillus species.

According to a first aspect of the invention, an antibody is described which binds to anthrax. Preferably the antibody binds specifically to the Bacillus anthracis protein EA1.

More preferably, the antibody comprises at least one of amino acid sequences:

### SEQ ID No 17;

### SEQ ID No. 10;

### SEQ ID No. 18;

### SEQ ID No. 19;

### SEQ ID No. 4;

### SEQ ID No. 13;

### SEQ ID No. 20;

### SEQ ID No. 14 or

SEQ ID No. 21 or a variant of one such sequence.

The expression “variant” as used in relation to amino acid sequences refers to such sequences which differ from the base sequence from which they are derived in that one or more amino acids within the sequence are substituted for other amino acids, but which retain the ability of the base sequence to encode polypeptides that are functionally equivalent to those defined by any of SEQ ID No.s 1-27, that is they encode polypeptides which bind to anthrax, preferrably via the EA1 protein. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will be at least 60% identical, preferably at least 75% identical, and more preferably at least 90% identical to the base sequence.

Identity in this instance can be judged for example using the BLAST program or the algorithm of Lipman-Pearson, with Ktuple:2, gap penalty:4, Gap Length Penalty:12, standard PAM scoring matrix (Lipman, D J. and Pearson, W. R., Rapid and Sensitive Protein Similarity Searches, Science, 1985, vol. 227, 1435-1441).

The term “fragment thereof” refers to any portion of the given amino acid sequence, which has the same activity as the complete amino acid sequence. Fragments will suitably comprise at least 5 and preferably at least 10 consecutive amino acids from the basic sequence.

In a further preferred embodiment of the invention, the hypervariable regions of the antibody are characterised thus:

CDR-L1 comprises SEQ ID No 17 or SEQ ID No. 10 or
CDR-L2 comprises SEQ ID No. 18 or
CDR-L3 comprises SEQ ID No. 19 or
CDR-H1 comprises SEQ ID No. 4 or SEQ ID No. 13 or
CDR-H2 comprises SEQ ID No. 20 or SEQ ID No. 14 or
CDR-H3 comprises SEQ ID No. 21

In another preferred embodiment, the antibody comprises SEQ ID No.s 1-16 or a variant thereof or a fragment thereof.

In a most preferred embodiment, the antibody comprises.

### SEQ ID No.s 1, 2, 3, 4, 5 and 6;

### SEQ ID No.s 7, 2, 3, 4, 8 and 6;

### SEQ ID No.s 7, 2, 3, 4, 5 and 6;

### SEQ ID No.s 9, 2, 3, 4, 5 and 6;

SEQ ID No.s 10, 11, 12, 13, 14 and 15 or,

### SEQ ID No.s 7, 2, 3, 4, 5 and 16;

According to a second aspect of the invention, a method of detecting anthrax comprises binding of an antibody according to the invention to anthrax spores.

A third aspect of the invention describes a nucleic acid encoding an antibody of the invention. Preferrably such a nucleic acid comprises any of SEQ ID No.s 28-39 or a variant of one of these.

The term “variant” in relation to a polynucleotide sequences means any substitution of, variation of, modification of, replacement of deletion of, or the addition of one or more nucleic acid(s) from or to a polynucleotide sequence providing the resultant protein sequence encoded by the polynucleotide exhibits the same properties as the protein encoded by the basic sequence. The term therefore includes alleleic variants and also includes a polynucleotide which hybridises to the basic polynucleotide sequence. Preferably, such hybridisation occurs at, or between low and high stringency conditions. In general terms, low stringency conditions can be defined as 3×SSC at about ambient temperature to about 55° C. and high stringency condition as 0.1×SSC at about 65° C. SSC is the name of the buffer of 0.15M NaCl, 0.015M tri-sodium citrate. 3×SSC is three times as strong as SSC and so on.

Antibodies according to the invention also have utility as medicaments for the treatment against infection by Bacillus anthracis and the manufacture of medicaments therefor.

The invention will now be described, by non-limiting example, with reference to the following figures in which:

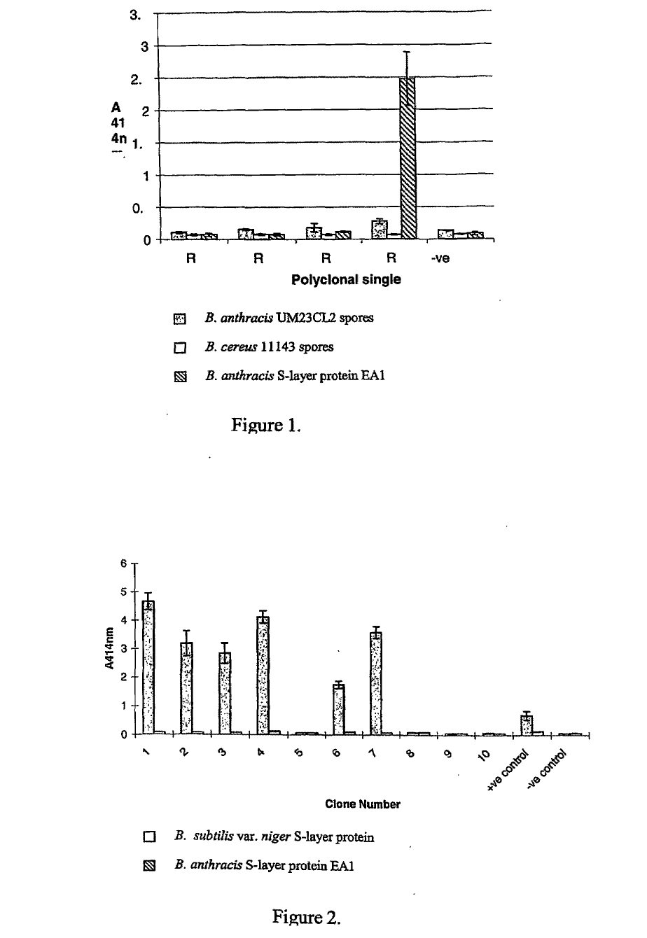

FIG. 1 illustrates ELISA results showing binding of polyclonal anti-EA1 scFv to EA1 from each round of the non-competitive biopanning procedure;

FIG. 2 shows ELISA results demonstrating binding of ten monoclonal scFv to B. anthracis EA1 and to B. subtilis var. niger S-layer protein;

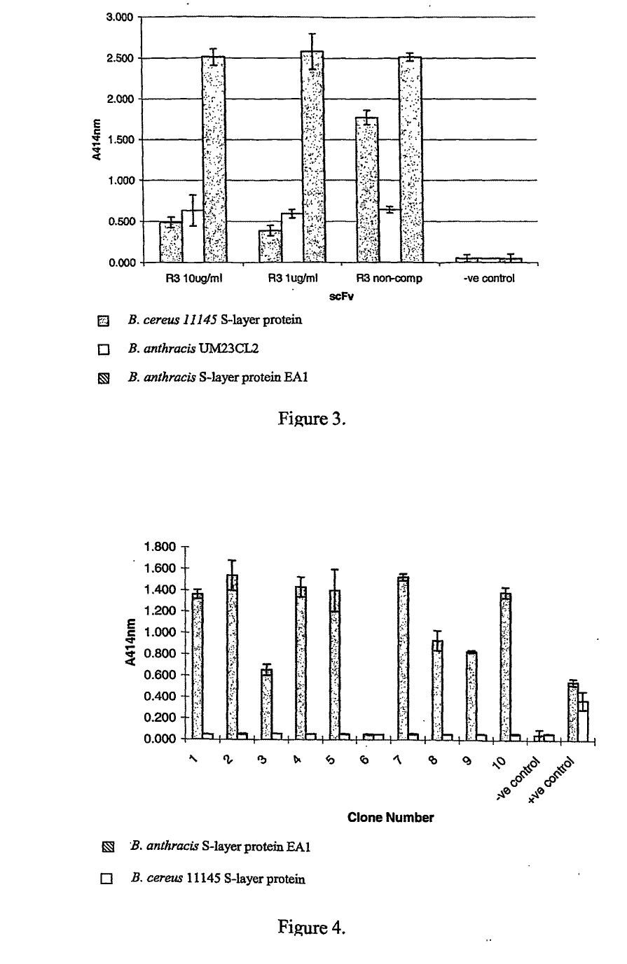

FIG. 3 shows ELISA results illustrating binding of polyclonal anti-EA1 from round 3 of competitive and non-competitive biopanning procedures;

FIG. 4 shows an example of ELISA results showing binding of anti-EA1 monoclonal scFvs selected by a competitive biopanning procedures that show no cross reactivity with B. cereus 11145 S-layer protein;

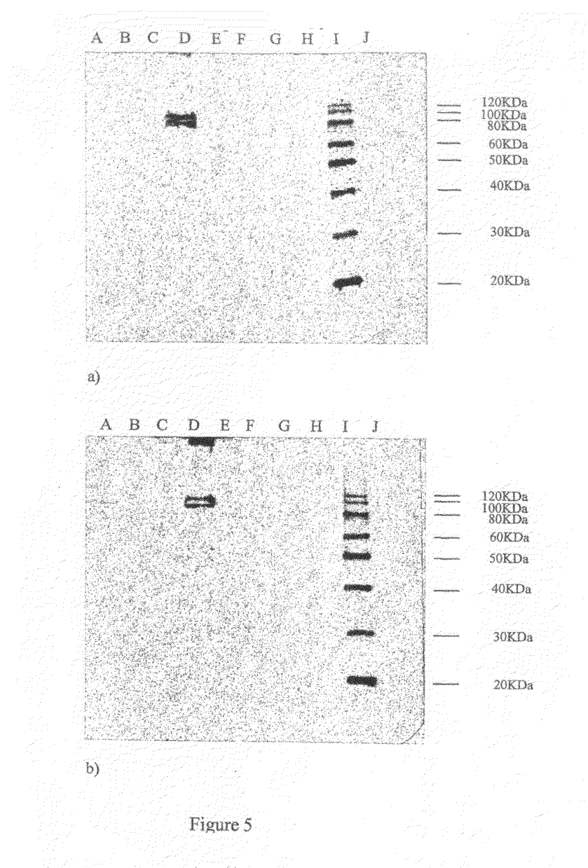

FIG. 5 shows Cross reactivity of monoclonal scFv selected by competitive biopanning to Bacillus S-layer proteins by Western blot and

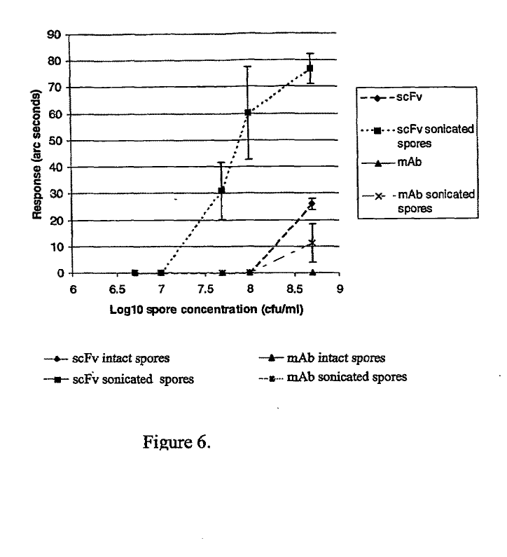

FIG. 6 shows Detection of B. anthracis UM23CL2 spores on a real time biosensor using a competitively selected scFv and a monoclonal anti-EA1 antibody.

### MATERIALS AND METHODS

### Bacterial Strains and Plasmids

Strain RBA91 (PXO1⁻, PXO2⁻B. anthracis Sap mutant) was provided by the Pasteur Institute (25,28 rue du Docteur Roux, Paris). B. cereus (NCTC 11143, NCTC 9946 and NCTC 11145), B. pumilus (NCTC 10337), B. brevis (NCTC 2611), B. coagulans (NCTC 10334), B. subtilis var. niger (NCTC 10073), B. thuringiensis var. kurstaki and B. thuringiensis var. israelensis were obtained from NCTC (PHLS, 61 Colindale Avenue, London). Plasmids pAK100 (used for phage display) and pAK300 (used for production of soluble scFv) were a kind gift from Dr A. Plückthun (University of Zürich, Switzerland), and were used as previously described by Krebber et al., (Reference 13).

### Preparation of S-Layer Proteins

50 ml bacterial cultures were grown overnight at 37° C. in SPY medium. Cultures were centrifuged at 8000 g for 30 minutes at 4° C. and resuspended in 5M guanidine hydrochloride 50 mM Tris-HCl pH 7.2. The resuspended pellets were incubated for 2 h at 20° C. with shaking, then centrifuged at 6000 g for 10 minutes at 4° C. The supernatant was removed and dialysed against 4 l of 50 mM Tris-HCl pH 7.5 overnight at 4° C. S-layer self-assembly products were sedimented by centrifugation for 30 min at 4° C. The precipitate and supernatant were analysed by SDS-PAGE to confirm the presence of S-layer proteins. The soluble S-layer protein contained in the supernatant was concentrated by ultrafiltration and filtered using a 0.45 μm filter. An aliquot of protein before and after concentration was retained for analysis. Further purification of S-layer proteins was achieved using a HiLoad 1660 Superdex 200 preparatory grade column (Amersham, Amersham Place, Little Chalfont, Buckinghamshire, UK) and an AKTA FPLC system (Amersham, Amersham Place, Little Chalfont, Buckinghamshire, UK), monitoring purity by SDS-PAGE. Fractions that contained pure S-layer protein were pooled and the concentration determined by BCA Assay (Pierce, Century House, High Street, Tattenhall, Cheshire, UK), according to the manufacturers instructions

### Spore Production

Spores were prepared using New Sporulation agar (3.0 g/litre Difo Tryptone; 6.0 g/litre Oxoid bacteriological peptone; 3.0 g/litre Oxoid yeast extract: 1.5 g/litre Oxoid Lab Lemco; 1 ml 0.1% MnCl₂.4H₂O; 25 g/litre Difco Bacto agar) or isolation agar (6.0 g/litre Oxoid nutrient broth n=2; 0.3 g/litre MnSO₄.H₂O; 0.25 g/litre KH₂PO₄; 12.0 g/litre oxoid technical agar n=3) and incubated at 37° C. until the cultures contained >95% phase bright spores. The spores were harvested by release from the solid media using ice cold sterile distilled water and subsequently centrifuged at 10000 g for 10 minutes at 4° C. and then washed 10 times in ice cold sterile distilled water to remove vegetative cells and debris. Preparations were examined using phase contrast microscopy to confirm that the preparations contained >95% phase bright spores.

Construction and Use of Immune Mouse scFv Library

Six 12 week old female Balb/c mice were immunised with irradiated B. anthracis Ames spores. Each immunisation consisted of 1×10⁷ spores in Freunds incomplete adjuvant. Mice were immunised 4 times at intervals of three weeks, and killed by cervical dislocation once they showed a sufficiently high titre (>1:100 000) to the spores by endpoint ELISA. Spleens were removed from the killed mice and splenic mRNA isolated using Trizol reagent (Invitrogen, Fountain Road, Inchinnan Business Park, Paisley, UK.). The total RNA from the immunised mice was used to produce the immune scFv library, PCR amplification of antibody sequences, overlap extension PCR, cloning of the assembled scFv sequence into pAK100 and production of phage-displayed scFv was carried out as described by Krebber et al. (Reference 13).

Biopanning with EA1

Immunotubes (Nunc, BRL, Life Technologies Ltd., Trident House, Washington Road, Paisley, UK) were coated with 1 ml of purified EA1 at 10 μg/ml in PBS overnight at 4° C. and blocked with 2% (w/v) milk powder PBS (MPBS). 100 μl of scFv-phage were mixed with 900 μl MPBS, incubated for one hour at room temperature, and added to the coated immunotubes. After a 2 hour room temperature incubation the immunotubes were washed 10 times with PBS 0.1% (v/v) Tween 20. Bound phage were eluted with 100 mM triethylamine and neutralised with 500 μl 1 M Tris HCl pH 7.5. Eluted phage were infected into log phase XL1-Blue E. coli, and plated on a 24 cm square 2×YT 1% (w/v) glucose 30 μg/ml chloramphenicol agar plate and incubated overnight at 30° C. This procedure was repeated for each round of panning carried out. Competitive panning was carried out in an identical fashion, adding S-layer extracts to the scFv-phage MPBS solution for 1 hour before panning. The concentrations of antigen used for competitive panning were 50 μg/ml B. cereus 11145 S-layer protein, and 25 μg/ml of B. cereus 11143, B. cereus 9946 and B. pumilus S-layer protein.

DNA Fingerprinting of scFv Clones

scFv sequences from selected clones were amplified by PCR primers surrounding the scFv sequence (scfor and scback; (Reference 16)) and subjected to BstN1 digestion to determine the diversity of the original library and each consecutive round of selection. Restriction digest products were resolved on 4% E-gels (Invitrogen), using 25 bp markers. PCR and DNA fingerprinting were carried out on 10 randomly selected scFv from rounds 1 and 2 and 50 scFv clones from round three.

Preparing DNA for Automated Fluorescent DNA Sequencing of Round 3 scFv

Plasmid preps were completed using the Mini Plasmid Spin Prep Kit (Qiagen), according to the manufacturers instructions. The scFv DNA was sequenced to confirm that clones were unique. These sequences were used for further analysis. The scFv were sequenced using forward and reverse primers for the plasmid (Oswell, UK or MWG, Sweden). Sequencing data from the DNA of the scFv was translated to a protein sequence using the Expasy translation tool. (http://ca.expasy.org/tools/dna.html). The correct reading frame was initially identified through the presence of GGGGS and consecutive repeats of this sequence required for the linker sequence between the VH and VL regions that make up the scFv. Heavy and light chain CDRs were identified using the Kabat definition, as described by Martin (Reference 36). The heavy and light chain CDR sequences were compared for the scFv sequences and similarities identified.

### ELISA

100 μl of EA1, S-layer extract (10 μg/ml in PBS) or Bacillus spores (1×10⁶ spores/ml in water) and appropriate control antigens were coated onto Immulon2 plates (Nunc.) and incubated overnight at 5° C. PEG-purified phage-displayed scFv were diluted with MPBS, and an anti-M13 HRP conjugated antibody (Sigma, Fancy Road, Poole, UK) used to detect bound phage. Bound phage were quantified by measuring the conversion of ABTS substrate to coloured product based on A405 readings in an automated ELISA reader (Anthos 2001, Anthos Labtec Instruments, Salzburg, Austria).

### Western Blot Analysis

Proteins were prepared in LDS sample buffer (Invitrogen) according to the manufacturers instructions and separated by SDS-PAGE in MES buffer (Invitrogen) using precast 10% NuPAGE® BIS-TRIS gels at 200V for 40 mins. Proteins were then blotted onto 0.2 μm nitrocellulose membranes in NUPAGE® transfer buffer (Invitrogen) at 30V for 1.5 hrs. For determination of molecular mass MagicMark™ Western protein standard (Invitrogen) was used. Blots were rinsed briefly PBST in (PBS 0.1% Tween 20 v/v) and incubated in 5% milk powder PBST overnight at 4° C. Blots were washed 1×15 mins then 2×5 mins in PBST and incubated with the appropriate scFv antibody at 2.5 μg/ml in 3% (w/v) milk powder PBST for 1 h at room temperature with shaking. Blots were washed as described previously and incubated in an anti-HIS HRP conjugate (Sigma) at 1:1000 and an anti-rabbit HRP conjugate (Amersham) antibody at 1:1000 dilution in 3% (w/v) milk powder PBST for 1 h at room temperature with shaking. Blots were washed 1×15 mins then 4×15 mins in PBST and protein bands recognised by the antibody were visualised by enhanced chemiluminescence (ECL Detection reagent kit, Amersham BioSciences, Chalfont, Bucks, UK.) and exposed to ECL hyperfilm (Amersham BioSciences, Chalfont St. Giles, Bucks, UK).

Kinetic Analysis of scFv Binding

The kinetic data for scFv binding purified EA1 was obtained using the BIAcore 3000 (BIAcore, Rapsgatan 7, Uppsala, Sweden) with EA1 immobilised onto a CM5 sensor chip, and B. cereus 11145 S-layer protein as a negative control. Approximately 1500 RU was immobilised onto the surface using standard amine coupling and unreacted sites blocked with 1M ethanolamine pH 8.5. ScFv were passed over the immobilised protein at concentrations varying from 5-400 nM in HBS EP buffer at a flow rate of 10 μl per minute. To examine cross reactivity, the antibody was immobilised onto a surface, and S-layer proteins from B. cereus 11145, B. cereus 11143, B. cereus 9946, B. thuringiensis var. israelensis, thuringiensis var. kurstaki, B. pumilus, B. brevis, B. coagulans and B. sutilis var. niger passed over at final concentration of 400 nM.

### Detection of Whole Spores and Evaluation of Sensitivity Using an Optical Biosensor

The Resonant Mirror (RM, Thermo Labsystems, Saxon Way, Bar Hill, Cambridge, UK.) was used to demonstrate detection of whole B. anthracis spores. Antibodies were immobilised onto a RM T70 low molecular weight carboxymethylated dextran (CMD) cuvette surface by standard EDC/NHS coupling methods. The spores were passed over at various concentrations for 10 minutes and the surface regenerated using 20 mM KOH for 3 minutes.

### Results & Discussion

### Single Chain Antibodies Produced by Non-Competitive Panning

Referring to FIG. 1, the non-competitive panning strategy used here required three rounds of biopanning against an immobilised target antigen, EA1. The observed binding to EA1 in ELISA by the polyclonal population of scFv present after each round of selection showed an increasing signal after round 2 and 3 of selection. The immune library used was expected to contain antibodies to EA1 as the mice had been immunised with B. anthracis spores. This library would be expected to contain scFv to a range of spore and exosporium antigens, and indeed has been used to produce antibodies to several other spores surface proteins in addition to EA1 (data not shown). The large increase in signal observed after round 3 is likely to be linked to the increased stringency of washing in this round (20 washes) compared with the lower stringency of the earlier rounds (10 washes).

Polyclonal phage-displayed scFv from each round of the biopanning procedure (unpanned library designated round 0; R0-R3 represents the results of each panning round) were diluted 50% (v/v) in MPBS for detection of EA1. An anti-ovalbumin scFv was used as a negative control (labelled −ve control). Bound phage were detected using an anti-M13 HRP conjugated antibody. Assays were performed in triplicate; error bars show two standard deviations from the mean. A positive result was defined as being higher than the average of the background signal plus three standard deviations of the mean background sample.

After round 3 of non-competitive biopanning 50 scFv clones were selected and their ability to bind EA1 assessed by direct ELISA. A sample result from ten of these clones is shown in FIG. 2. Of the 50 scFv clones analysed 43 were found to bind to EA1, with 7 clones not showing any binding to EA1. Only 2 scFv clones demonstrated any cross reactivity to B. subtilis var niger S-layer protein, with the remaining 41 showing no detectable cross reactivity to B. subtilis var niger S-layer protein. Unique clones were identified by BstN1 fingerprinting all the scFv clones that bound to EA1, demonstrating 13 unique clones. These clones were all confirmed to be different by DNA sequencing (data not shown), and are referred to here as scFv1 to scFv 13.

Monoclonal phage-displayed scFv isolated from the third round of EA1 panning were prepared from 2 ml of supernatant by PEG precipitation and resuspended in 0.4 ml 2% (w/v) MPBS. An anti-ovalbumin scFv was used as a negative control (labelled −ve control), and a polyclonal scFv known to bind to B. anthracis spores were used as a positive control (labelled +ve control). Bound phage were detected using an anti-M13 HRP conjugated antibody. Assays were performed in triplicate, error bars show two standard deviations from the mean. A positive result was defined as being higher than the average of the background signal plus three standard deviations of the mean background sample.

A selection of Bacillus species were tested for cross reactivity. An organism of particular concern was B. thuringiensis, used (and sprayed) widely as an insecticide. BLAST searches demonstrate a very high degree of similarity between the EA1 protein sequence of B. thuringiensis and B. anthracis. The other organism of concern was B. cereus, another closely related species (Reference 6).

The cross reactivity of the 13 unique scFv was determined by direct ELISA against S-layer proteins isolated from other Bacillus species. All of the 13 unique scFv tested showed cross reactivity with S-layer proteins from the other Bacillus tested (for brevity these ELISA results are summarised in Table 1). The highest degree of cross reactivity was observed with B. cereus NCTC 11145 and B. pumilus S-layer proteins.

TABLE 1
Summary of ELISA results demonstrating the binding of unique monoclonal
scFv to EA1 and the cross reactivity to other S-layer proteins, determined by ELISA.

| | | | | | | | | | | B. anthracis |
|---|---|---|---|---|---|---|---|---|---|---|
| | | B. thuringiensis | B. thuringiensis | B. cereus | B. cereus | | | | | UM23CL2 |
| | EA1 | var. israelensis | var. kurstald | 11143 | 9946 | B. cereus 11145 | B. pumllus | B. brevis | B. coagulans | spores |
| scFv 1 | +++ | − | − | − | ++ | − | − | − | − | ++ |
| scFv 2 | +++ | − | − | − | − | +++ | +++ | − | − | ++ |
| scFv 3 | +++ | − | − | − | − | +++ | +++ | − | − | ++ |
| scFv 4 | +++ | − | − | − | − | ++ | ++ | − | − | ++ |
| scFv 5 | +++ | − | − | − | − | +++ | ++ | − | − | ++ |
| scFv 6 | +++ | − | ++ | ++ | ++ | +++ | +++ | ++ | − | ++ |
| scFv 7 | +++ | − | − | − | ++ | +++ | ++ | − | − | ++ |
| scFv 8 | + | − | − | − | − | +++ | +++ | − | − | ++ |
| scFv 9 | +++ | − | − | − | − | ++ | − | − | − | + |
| scFv 10 | + | − | − | − | − | +++ | +++ | − | − | ++ |
| scFv 11 | + | − | − | − | − | − | +++ | − | − | ++ |
| scFv 12 | +++ | − | − | +++ | − | − | − | − | − | ++ |
| scFv 13 | + | − | − | ++ | − | +++ | − | − | − | ++ |
| EA1.1 | +++ | − | − | − | − | − | − | − | − | ++ |
| EA1.23 | +++ | − | − | − | − | − | − | − | − | ++ |
| EA1.10 | +++ | − | − | − | − | − | − | − | − | + |
| EA1.20 | +++ | − | − | − | − | − | − | − | − | ++ |
| EA10.1 | +++ | − | − | − | − | − | − | − | − | ++ |
| EA10.4 | +++ | − | − | − | − | − | − | − | − | ++ |

Soluble scFv were produced, purified by IMAC and used at 5 μg/ml in ELISA. Bound phage were detected using an anti-his tag HRP conjugated antibody (Sigma). Each assay was performed in triplicate. A summary of these ELISA assays is presented here; results are expressed in terms of the percentage of the maximum signal seen in the assay (+++ indicates 60-100% of the maximum, ++ indicates 20-59%, + indicates a signal greater than detection threshold, defined as the background signal plus 3 standard deviations from the mean of the background signal, and − indicates a signal below the detection threshold.)

The high cross reactivity of the scFv produced by non-competitive biopanning suggests that the S-layer proteins of these species may contain proteins that are homologous to the B. anthracis EA1 S-layer protein. The higher degree of cross reactivity observed with B. pumilus, B. subtilis var. niger and B. cereus 11145 suggests that these proteins demonstrate the greatest degree of similarity to EA1 or that a highly conserved or immunodominant epitope exists within these species (Reference 7). Irrespective of the mechanism, none of these anti-EA1 scFvs were of any use for specific detection of B. anthracis.

### Single Chain Antibodies Produced by Competitive Panning

In order to isolate B. anthracis specific anti-EA1 scFv a competitive panning strategy was adopted. This involved negative selection of cross-reacting scFv by binding them to S-layer proteins from species that cross-reacted with our original anti-EA1 scFvs. The panning procedure was repeated as detailed for the non-competitive strategy, but this time a mixture of competitive S-layer extracts (50 μg/ml B. cereus 11145 S-layer protein, and 25 μg/ml of B. cereus 11143, B. cereus 9946 and B. pumilus S-layer protein) were added to the solution of panning phage at the first panning round. The amount of EA1 used to coat the immunotube was also varied (1 or 10 μg/ml). Binding to EA1 in ELISA by the polyclonal population of scFv present after each round of panning showed a large increase in signal after the first round of panning. This could be due to the stringent negative selection in the first round that would have decreased the number of scFv that may bind to EA1. There was no significant difference in signal observed between the polyclonal scFvs selected using 1 or 10 μg/ml of EA1 (FIG. 3). After round 3 there was still some cross reactivity with B. cereus 11145 S-layer protein, however this was much lower as that observed in round 3 scFv phage using the non-competitive approach (FIG. 3).

Polyclonal phage-displayed scFv from round 3 of the biopanning procedure were diluted 50% (v/v) in MPBS for detection of B. anthracis EA1, B. cereus 11145 S-layer protein and B. anthracis UM23CL2 spores. Three biopanning procedures were used; competitive panning with B. anthracis EA1 at 10 μg/ml (labelled R3 10 μg/ml) or 1 μg/ml (labelled R3 1 μg/ml), or non-competitive panning with B. anthracis EA1 at 10 μg/ml (labelled R3 non-comp). An anti-ovalbumin scFv was used as a negative control (labelled −ve control). Bound phage were detected using an anti-M13 HRP conjugated antibody. Assays were performed in triplicate, error bars show two standard deviations from the mean. A positive result was defined as being higher than the average of the background signal plus three standard deviations of the mean background sample.

These ELISA results that indicate some scFvs that cross react with B. cereus 11145 S-layer protein are still selected, even though a competitive selection procedure was used. This may be because the cross reactive antigens from closely related species were sufficiently in excess (although 10 fold excess was used) leaving cross-reactive scFv to bind to the target. Furthermore, the cross reactive epitope within the S-layer protein that binds to the scFv may be immunodominant, ensuring that a large proportion of the scFv will bind to this site (Reference 7). It may also be due to selective pressures imposed by growth or expression, or a combination of these factors. Despite the cross reactivity of the round 3 polyclonal scFv population it believed that some scFv clones with reduced cross reactivity would have been selected.

After round 3 of competitive panning 50 scFv (25 from each of the 1 μg/ml and 10 μg/ml selections) clones were selected and analysed by ELISA. Ten of these are shown as an example in FIG. 4. In total, 18 scFv cross-reacted with B. cereus 11145 S-layer protein (6 from the 1 μg/ml strategy and 12 from the 10)g/ml strategy). Three of the selected scFv did not bind EA1, all of which were taken from the 1 μg/ml EA1 panning. 29 of the 50 scFv analysed were found to be specific for B. anthracis EA1; 16 of these were selected using 1 μg/ml EA1 and 13 were selected using EA1 at a concentration of 10 μg/ml. This result demonstrates the utility of the competitive panning method. It was not possible to obtain scFv antibodies specific to B. anthracis EA1 by conventional non-competitive panning, while the competitive method rapidly isolated non cross-reactive scFv antibodies that would not recognise B. cereus 11145 S-layer protein.

Monoclonal phage-displayed scFv isolated from the third round of EA1 competitive panning were prepared from 2 ml of supernatant by PEG precipitation and resuspended in 0.4 ml 2% (w/v) MPBS. An anti-ovalbumin scFv was used as a negative control (labelled −ve control), and a polyclonal scFv known to bind to B. anthracis spores were used as a positive control (labelled +ve control). Bound phage were detected using an anti-M13 HRP conjugated antibody. Assays were performed in triplicate, error bars show two standard deviations from the mean. A positive result was defined as being higher than the average of the background signal plus three standard deviations of the mean background sample.

ScFv antibodies from rounds 1 and 3 of the competitive panning were examined by BstN1 fingerprinting to identify unique clones. As expected there was greater diversity of scFv after round 1 than after round 3 of panning. However, the diversity of scFv was much lower using the non-competitive strategy when compared with the scFv isolated by the competitive strategy. Only six unique scFv were identified in total from both the 1 μg/ml and 10 μg/ml competitive panning strategies by BstN1 fingerprinting, and were confirmed unique by DNA sequence analysis (EA1.1, EA1.23, EA1.10, EA1.20, EA10.1, EA10.4; data not shown). This suggests that competitive panning rapidly lead to the elimination of a large percentage of the population of scFv clones by negative selection. This could suggest that some binders were lost through further rounds of selection, either due to low affinity or growth or expression selection pressures. The scFvs produced by the competitive approach showed less diversity in the CDRs than those produced by competitive selection. As expected, variations in CDR-H3 gave the main source of diversity between the different antibodies, as CDR-H3 is mainly responsible for specificity (Reference 17).

The cross reactivity of the six unique scFv isolated by competitive panning was determined by direct ELISA against S-layer proteins isolated from other Bacillus species. None of these scFv showed any cross reactivity with S-layer proteins from the other Bacillus tested (Table 1). This indicates that all six scFv recognise epitopes that are unique to B. anthracis EA1. To verify this result the scFvs were used to probe purified Bacillus S-layer extracts on Western blots. Detection of purified B. anthracis EA1 was demonstrated with all six scFv generated (two shown as examples in FIG. 5). EA1.1, EA1.23, EA1.10 and EA10.1 showed no cross reactivity with any other Bacillus S-layer proteins tested, even in grossly overexposed blots. EA1.20 and EA10.4 did show low levels of cross reactivity with B. pumilus S-layer protein (only visible after a 30 minute exposure; example shown in FIG. 5b). This cross reactivity with B. pumilus S-layer protein was never observed by ELISA, Resonant Mirror or BIAcore analysis (data not shown).

The cross reactivity of two different antibodies is shown here; scFv EA1.1 (blot a) and scFv EA10.4 (blot b). 5 μg of each S-layer protein extract was run per lane on a 10% BIS TRIS NuPAGE gels (Invitrogen) and blotted onto nitrocellulose membranes (Invitrogen). Lane loading was as follows: A) B. pumilus S-layer protein, B) B. brevis S-layer protein, C) B. coagulans S-layer protein, D) B. anthracis S-layer protein EA1, E) B. cereus 11145 S-layer protein, F) B. cereus 11143 S-layer protein, G) B. cereus 9946 S-layer protein H) B. subtilis var. niger S-layer protein I) MagicMark™ Western standard, J) ovalbumin negative control. After probing, bound scFv was visualised using an anti-his tag HRP conjugated antibody (Sigma) followed by enhanced chemiluminescent detection.

### Use of Specific EA1 Antibodies on Biosensors

Analysis of the affinity constants of the six B. anthracis EA1-specific scFv on the BIAcore biosensor demonstrated that the highest affinity constants were seen in the scFv selected using 1 μg/ml of EA1 compared to those isolated using 10 μg/ml EA1 (Table 2). No binding was observed on the BIAcore with any of these six specific scFv for any S-layers evaluated (B. pumilus, B. cereus 11145, B. cereus 11143, B. cereus 9946, B. coagulans, B. brevis, B. subtilis var. niger, B. thuringiensis var. israelensis and B. thuringiensis var. kurstaki; data not shown).

TABLE 2
Equilibrium association (KA) and dissociation (KD)
constants for scFv antibody clones produced using
competitive panning strategies.

| Single chain | KA (1/M) | KD (M) |
|---|---|---|
| EA1.1 | 5.85E+10 | 1.71E−11 |
| EA1.23 | 4.48E+10 | 2.23E−11 |
| EA1.10 | 1.72E+08 | 5.81E−09 |
| EA1.20 | 1.89E+10 | 5.28E−11 |
| EA10.1 | 1.01E+09 | 9.91E−10 |
| EA10.4 | 3.55E+08 | 2.81E−09 |

Equilibrium constants were derived using the BIAevaluation software (Biacore) using a simple Langmuir 1:1 binding model and the association (Kₐ) and dissociation (K_{d}) rate constants calculated for each set of data. The equilibrium constant KA was calculated from the ratio Kₐ/K_{d} and KD from K_{d}/Kₐ.

These results revealed that these B. anthracis specific scFvs had apparent nanomolar affinities for EA1; very satisfactory values for antibodies to be used for sensitive detection. As absolute specificity was used as the original criteria for success, other stronger binders (albeit cross-reactive) may have been eliminated through the competitive panning process. It is likely that the affinities of these antibodies could be improved by further maturation techniques if desired (for example, References 10 and 20).

Demonstration of the detection of intact B. anthracis spores was carried out using the Resonant Mirror (Thermo Labsystems) biosensor. Three of the specific scFvs (EA1.1, EA1.23 and EA1.10) were taken forward for evaluation of the sensitivity to whole B. anthracis UM23C12 spores on this real-time biosensor. For comparison an anti-EA1 monoclonal antibody was also evaluated. All scFv demonstrated detection of untreated B. anthracis spores (FIG. 6), although the sensitivity of the assay improved significantly when the spores were sonicated. In comparison the mAb could not detect intact B. anthracis UM23CL2 spores at any of the concentrations tested, and only a small amount of binding was observed after sonication (FIG. 6). The scFv showed no cross reactivity to any other Bacillus species spores tested (both untreated or sonicated) while the monoclonal antibody showed detection of sonicated B. cereus 11145 spores using this method.

Antibodies were immobilised using standard EDC/NHS coupling onto a T70 CMD surface (Labsystems, Affinity sensors). Intact or sonicated spores were passed over immobilised scFv EA1.1 (scFv intact or sonicated spores) or anti-EA1 monoclonal (mAb intact or sonicated spores) antibody in PBS 0.05% (v/v) Tween 20. Spores were sonicated as described previously (Reference 25).

### Competitive Biopanning: A Significant Advantage?

scFv libraries are routinely produced from immunised mice in order to obtain scFvs that show high affinities for the desired targets. While naive libraries have been used successfully, higher affinities and a wider diversity of antibodies have been obtained from an immune library. Competitive panning has proved extremely useful in this case to reduce the complexity of these immune libraries by eliminating cross-reactive antibodies. Immune libraries have the advantage of having undergone significant affinity maturation in the mouse; the antibodies evolve in vivo in the B cell by hypersomatic mutation within the hypervariable regions to enhance specificity and affinity (Reference 1).

The immune library used here was created from mice immunised with a complex antigen (whole B. anthracis spores) not specifically against EA1, so this library would have contained a selection of antibodies that bound to a range of target antigens. The diversity of this library may be limited with respect to those that recognise a range of EA1 epitopes. If an anti-EA1 library had been utilised, this may have enhanced the probability of isolating an EA1 specific scFv by non-competitive selection, although in practice we find that producing a single library for each complex target allows a range of antibodies to be isolated while minimising animal use. EA1 is a major antigen associated with the vegetative cells of B. anthracis (Reference 18). Western blot analysis of a polyclonal goat antiserum raised against whole B. anthracis spores also showed binding to EA1, demonstrating that it is present and immunogenic in spore preparations (data not shown). It is important to remember that a proportion of the specific EA1 antibodies must have been lost during non-competitive panning, perhaps due to competition from non-specific antibodies with higher affinities for the target or other selection pressures such as slow growth.

The use of a competitive panning procedure (sometimes termed pre-adsorption, subtractive antibody screening or negative selection) has also been used for other targets; Krebs et al. (Reference 14) described a method by which pre- and post-adsorption steps could be utilised to select out scFv that bound to cross reactive targets to produce specific scFv. Specific anti-melanoma antibodies have also been prepared using by pre-absorbing with melanocytes 10 times (Reference 2). The much simpler procedure of one step negative selection demonstrated here shows that a complex mixture of competitors can be used in the first round of selection to remove the majority of non-specific binders and isolate a number of scFvs specific for the original target.

The generation of specific recognition elements for EA1 and consequently B. anthracis spores in this case was only possible using the competitive strategy. The resultant recombinant antibodies can be used successfully across a wide range of detection techniques, from the conventional laboratory analysis methods such as ELISA and Western blotting to sophisticated evanescent wave based biodetectors that can be used in the field. When used on the Resonant Mirror biosensor the sensor was able to signal specific detection of anthrax spores in real time, with no false positives even when exposed to very high backgrounds of closely related Bacillus species that commonly cross react with anti-anthrax polyclonal and monoclonal antibodies. Non-recombinant antibodies that are able to perform to this high specification are unknown to the inventors and this method of competitive panning gives a major advantage over conventional monoclonal and polyclonal approaches, especially for critical diagnostic and detection applications.

Table 3 lists the oligonucleotide sequences for CDRs of the six B. anthracis EA1-specific scFv antibodies along with various generic sequence formula derived from grouping certain sequences having common features. Table 4 shows the full antibody sequences.

TABLE 3

| | | | |
|---|---|---|---|
| | | | SEQ |
| | | | ID |
| Antibody | CDR | Sequence | No. |

| | | | |
|---|---|---|---|
| EA1.1 | L1 | AASKSVTTSGYSYMH | 1 |
| EA1.1 | L2 | LASNLES | 2 |
| EA1.1 | L3 | QHSRDLPWT | 3 |
| EA1.1 | H1 | SFGMH | 4 |
| EA1.1 | H2 | YISSDGSTIYYADTV | 5 |
| EA1.1 | H3 | WLGGYAMDY | 6 |
| EA1.10 | L1 | RASKSVTTSGYSYMH | 7 |
| EA1.10 | L2 | LASNLES | 2 |
| EA1.10 | L3 | QHSRDLPWT | 3 |
| EA1.10 | H1 | SFGMH | 4 |
| EA1.10 | H2 | YISSDLSTIYYADTV | 8 |
| EA1.10 | H3 | WLGGYAMDY | 6 |
| EA1.20 | L1 | RASKSVTTSGYSYMH | 7 |
| EA1.20 | L2 | LASNLES | 2 |
| EA1.20 | L3 | QHSRDLPWT | 3 |
| EA1.20 | H1 | SFGMH | 4 |
| EA1.20 | H2 | YISSDGSTIYYADTV | 5 |
| EA1.20 | H3 | WLGGYAMDY | 6 |
| EA1.23 | L1 | ASKSVTTSGYSYMH | 9 |
| EA1.23 | L2 | LASNLES | 2 |
| EA1.23 | L3 | QHSRDLPWT | 3 |
| EA1.23 | H1 | SFGMH | 4 |
| EA1.23 | H2 | YISSDGSTIYYADTV | 5 |
| EA1.23 | H3 | WLGGYAMDY | 6 |
| EA10.1 | L1 | HASQNINVWLS | 10 |
| EA10.1 | L2 | KASNLHT | 11 |
| EA10.1 | L3 | QQGQSYPWT | 12 |
| EA10.1 | H1 | SHWIE | 13 |
| EA10.1 | H2 | EILPGSGSTNYNEKFKD | 14 |
| EA10.1 | H3 | RDYGNNSFDY | 15 |
| EA10.4 | L1 | RASKSVTTSGYSYMH | 7 |
| EA10.4 | L2 | LASNLES | 2 |
| EA10.4 | L3 | QHSRDLPWT | 3 |
| EA10.4 | H1 | SFGMH | 4 |
| EA10.4 | H2 | YISSDLSTIYYADTV | 5 |
| EA10.4 | H3 | WLGGYAMDYKEPQSPSP | 16 |

TABLE 4

| | |
|---|---|
| Antibody | Sequence |

| | |
|---|---|
| EA1.1 | DYKDIVMTQSPASLLVSPGQRATISCAASKSVTTSGYSYMHW |
| | YQQKPGQPPKLLIYLASNLESGVPARFSGSGSGTDFTLNIHP |
| | VEEEDAATYYCQHSRDLPWTFGGGTKLEIKRGGGGSGGGGSE |
| | VKLVESGGGLVKPGGSLKLSCAASGFTFSSFGMHWVRQAPEK |
| | GLEWVAYISSDGSTIYYADTVKGRFTMSRDNPKNTLFLQMTS |
| | LRSEDTAMYYCVRWLGGYAMDYWGQGTSVT |
| | (SEQ ID No 22) |
| EA1.10 | DYKDIVMTQSPASLLVSPGQRATISCRASKSVTTSGYSYMHW |
| | YQQKPGQPPKLLIYLASNLESGVPARFSGSGSGTDFTLNIHP |
| | VEEEDAATYYCQHSRDLPWTFGGGTKLEIKRGGGGSGGGGSE |
| | VKLVESGGGLVKPGGSLKLSCAASGFTFSSFGMHWVRQAPEK |
| | GLEWVAYISSDLSTIYYADTVKGRFTMSRDNPKNTLFLQMTS |
| | LRSEDTAMYYCVRWLGGYAMDYWGQGTSVT |
| | (SEQ ID No 23) |
| EA1.20 | DYKDIVMTQSPASLLVSPGQRATISCRASKSVTTSGYSYMHW |
| | YQQKPGQPPKLLIYLASNLESGVPARFSGSGSGTDFTLNIHP |
| | VEEEDAATYYCQHSRDLPWTFGGLTKLEIKRGGGGSGGGGSE |
| | VKLVESGGGLVKPGGSLKLSCAASGFTFSSFGMHWVRQAPEK |
| | GLEWVAYISSDGSTIYYADTVKGRFTMSRDNPKNTLFLQMTS |
| | LRSEDTAMYYCVRWLGGYAMDYWGQGTSVT |
| | (SEQ ID No 24) |
| EA1.23 | DYKDIVMTQSPASLLVSPGQRATISCASKSVTTSGYSYMHWY |
| | QQKPGQPPKLLIYLASNLESGVPARFSGSGSGTDFTLNIHPV |
| | EEEDAATYYCQHSRDLPWTFGGGTKLEIKRGGGGSGGGGSEV |
| | KLVESGGGLVKPGGSLKLSCAASGFTFSSFGMHWVRQAPEKG |
| | LEWVAYISSDGSTIYYADTVKGRFTMSRDNPKNTLFLQMTSL |
| | RSEDTAMYYCVRWLGGYAMDYWGQGTSVTVSS |
| | (SEQ ID No 25) |
| EA10.1 | DYKDIQMIQSPSSLSASLGDTITITCHASQNINVWLSWYQQK |
| | PGNIPKLLIYKASNLHTGVPSRFSGSGSGTGFTLTISSLQPE |
| | DIATYYCQQGQSYPWTFGGGTKLEIKRGGGGSGGGGSGGGGS |
| | GGGGSEVQLQQSGAELMKPGASVKISCMATGYTFSSHWIEWV |
| | KQRPGHGLEWIGEILPGSGSTNYNEKFKDKATFTADTSSNTA |
| | YMQLISLTSEDSAVYYCARRDYGNNSFDYWGQGTTL |
| | (SEQ ID No 26) |
| EA10.4 | DYKDIVMTQSPASLLVSPGQRATISCRASKSVTTSGYSYMHW |
| | YQQKPGQPPKLLIYLASNLESGVPARFSGSGSGTDFTLNIHP |
| | VEEEDAATYYCQHSRDLPWTFGGGTKLEIKRGGGGSGGGGSE |
| | VKLVESGGGLVKPGGSLKLSCAASGFTFSSFGMHWVRQAPEK |
| | GLEWVAYISSDGSTIYYADTVKGRFTMSRDNPKNTLFLQMTS |
| | LRSEDTAMYYCVR WLGGYAMDYGVKEPQSPSP |
| | (SEQ ID No 27) |

Review of table 3 reveals that sequences for particular CDRs may be grouped together according to common features.

For example, SEQ ID no.s 1, 7 and 9, each of which represents CDR-L1 region of an antibody, include the common sequence ASKSVTTSGYSYMH (SEQ ID No 17);

Similarly SEQ ID No.s 2 and 11, each of which represents CDR-L2 region of an antibody, include the sequence ASN (SEQ ID No. 18);

SEQ ID No.s 3 and 12, each of which represents CDR-L3 region of an antibody, may be generally designated QXXXXPWT (SEQ ID No. 19) where X=an amino acid;

SEQ ID No.s 5 and 8, each of which represents CDR-H2 region of an antibody, may be generally designated YISSDXSTIYYADTV (SEQ ID No. 20) and

SEQ ID No.s 6, 15 and 16, each of which represents CDR-H3 region of an antibody, all contain the sequence XXXGXXXDY (SEQ ID No. 21).

Thus, each of the six antibodies falls within the general description:

CDR-L1 comprises SEQ ID No 17 or SEQ ID No. 10
CDR-L2 comprises SEQ ID No. 18
CDR-L3 comprises SEQ ID No. 19
CDR-H1 comprises SEQ ID No. 4 or SEQ ID No. 13
CDR-H2 comprises SEQ ID No. 20 or SEQ ID No. 14 and
CDR-H3 comprises SEQ ID No. 21
DNA Sequences for scFv

Each antibody was sequenced forward and reverse once, both translated using translation tool. The full amino acid sequence of the ScFv was identified by the presence of ‘GGGGS’ repeats denoting the linker sequence for the antibody. The translated forward and reverse polynucleotide sequences were used to compile the complete amino acid sequence.

EA1.1 Foward

(SEQ ID No 28)

TATGACCATGATTACGAATTTCTAGATAACGAGGGCAAATCATGAAATAC
CTATTGCCTACGGCAGCCGCTGGATTGTTATTACTCGCGGCCCAGCCGGC
CATGGCGGACTACAAAGATATTGTGATGACCCAATCTCCTGCTTCCTTAC
TTGTGTCTCCGGGGCAGAGGGCCACCATCTCATGCAGGGCCAGCAAAAGT
GTCACTACATCTGGCTATAGTTATATGCACTGGTACCAACAGAAACCAGG
ACAGCCACCCAAGCTCCTCATCTATCTTGCATCCAACCTAGAATCTGGGG
TCCCTGCCAGGTTCAGTGGCAGTGGGTCTGGGACAGACTTCACCCTCAAC
ATCCATCCTGTGGAAGAGGAGGATGCTGCAACCTATTACTGTCAGCACAG
TAGGGATCTTCCGTGGACGTTCGGTGGAGGCACCAAGCTGGAAATAAAAC
GTGGTGGTGGCGGCTCCGGTGGTGGTGGATCCGAGGTGAAGCTGGTGGAA
TCTGGGGGAGGCTTAGTGAAGCCTGGAGGGTCCCTGAAACTCTCCTGTGC
AGCCTCTGGATTCACTTTCAGTAGCTTTGGAATGCACTGGGTTCGTCAGG
CTCCAGAGAAGGGGCTGGAGTGGGTCGCATACATTAGTAGTGACGGTAGT
ACCATCTACTATGCAGACACAGTGAAAGGCCGATTCACCATGTCCAGAGA
CAATCCCAAGAACACCCTGTTCCTGCAAATGACCAGTCTAAGGTCTGAAG
ACACGGCCATGTATTACTGTGTAAGATG
EA1.1 reverse

(SEQ ID No 29)

GTAATACATGGCCGTGTCCTCAGACCTTAGACTGGTCATTTGCAGGAACA
GGGTGTTCTTGGGATTGTCTCTGGACATGGTGAATCGGCCCTTCACTGTG
TCTGCATAGTAGATGGTACTACCGTCACTACTAATGTATGCGACCCACTC
CAGCCCCTTCTCTGGAGCCTGACGAACCCAGTGCATTCCAAAGCTACTGA
AAGTGAATCCAGAGGCTGCACAGGAGAGTTTCAGGGACCCTCCAGGCTTC
ACTAAGCCTCCCCCAGATTCCACCAGCTTCACCTCGGATCCACCACCACC
GGAGCCGCCACCACCACGTTTTATTTCCAGCTTGGTGCCTCCACCGAACG
TCCACGGAAGATCCCTACTGTGCTGACAGTAATAGGTTGCAGCATCCTCC
TCTTCCACAGGATGGATGTTGAGGGTGAAGTCTGTCCCAGACCCACTGCC
ACTGAACCTGGCAGGGACCCCAGATTCTAGGTTGGATGCAAGATAGATGA
GGAGCTTGGGTGGCTGTCCTGGTTTCTGTTGGTACCAGTGCATATAACTA
TAGCCAGATGTAGTGACACTTTTGCTGGCCCTGCATGAGATGGTGGCCCT
CTGCCCCGGAGACACAAGTAAGGAAGCAGGAGATTGGGTCATCACAATAT
CTTTGTAGTCCGCCATGGCCGGCTGGGCCGCGAGTAATAACAATCCAGCG
GCCTGCCGTAAGCAATAGGTA
EA1.10 forward

(SEQ ID No 30)

GAAACAGCTATGACCATGATTACGAATTTCTAGATAACGAGGGCAAATCA
TGAAATACCTATTGCCTACGGCAGCCGCTGGATTGTTATTACTCGCGGCC
CAGCCGGCCATGGCGGACTACAAAGATATTGTGATGACCCAATCTCCTGC
TTCCTTACTTGTGTCTCCGGGGCAGAGGGCCACCATCTCATGCAGGGCCA
GCAAAAGTGTCACTACATCTGGCTATAGTTATATGCACTGGTACCAACAG
AAACCAGGACAGCCACCCAAGCTCCTCATCTATCTTGCATCCAACCTAGA
ATCTGGGGTCCCTGCCAGGTTCAGTGGCAGTGGGTCTGGGACAGACTTCA
CCCTCAACATCCATCCTGTGGAAGAGGAGGATGCTGCAACCTATTACTGT
CAGCACAGTAGGGATCTTCCGTGGACGTTCGGTGGAGGCACCAAGCTGGA
AATAAAACGTGGTGGTGGCGGCTCCGGTGGTGGTGGATCCGAGGTGAAGC
TGGTGGAATCTGGGGGAGGCTTAGTGAAGCCTGGAGGGTCCCTGAAACTC
TCCTGTGCAGCCTCTGGATTCACTTTCAGTAGCTTTGGAATGCACTGGGT
TCGTCAGGCTCCAGAGAAGGGGCTGGAGTGGGTCGCATACATTAGTAGTG
ACGGTAGTACCATCTACTATGCAGACACAGTGAAGGGCCGATTCACCATG
TCCAGAGACAATCCCAAGAACACCCTGTTCCTGCAAATGACCAGTCTAAG
GTCTGAGGACACGGCCATGTATTACTGTG
EA1.10 reverse

(SEQ ID No 31)

TGAGAGTGGTGCCTTGGCCCCAGTAGTCAAAGGAGTTATTACCGTAGTCC
CGTCTTGCACAGTAATAGACGGCAGAGTCCTCAGATGTCAGGCTGATGAG
TTGCATGTAGGCTGTGTTGGAGGATGTATCTGCAGTGAATGTGGCCTTGT
CCTTGAACTTCTCATTGTAGTTAGTACTACCACTTCCAGGTAAAATCTCT
CCAATCCACTCAAGGCCATGTCCAGGCCTCTGCTTTACCCACTCTATCCA
GTGGCTACTGAATGTGTAGCCAGTAGCCATGCAGGATATCTTCACTGAGG
CC
EA1.20 forward

(SEQ ID No 32)

GGAAACAGCTATGACCATGATTACGAATTTCTAGATAACGAGGGCAAATC
ATGAAATACCTATTGCCTACGGCAGCCGCTGGATTGTTATTACTCGCGGC
CCAGCCGGCCATGGCGGACTACAAAGATATTGTGATGACCCAATCTCCTG
CTTCCTTACTTGTGTCTCCGGGGCAGAGGGCCACCATCTCATGCAGGGCC
AGCAAAAGTGTCACTACATCTGGCTATAGTTATATGCACTGGTACCAACA
GAAACCAGGACAGCCACCCAAGCTCCTCATCTATCTTGCATCCAACCTAG
AATCTGGGGTCCCTGCCAGGTTCAGTGGCAGTGGGTCTGGGACAGACTTC
ACCCTCAACATCCATCCTGTGGAAGAGGAGGATGCTGCAACCTATTACTG
TCAGCACAGTAGGGATCTTCCGTGGACGTTCGGTGGAGGCACCAAGCTGG
AAATAAAACGTGGTGGTGGCGGCTCCGGTGGTGGTGGATCCGAGGTGAAG
CTGGTGGAATCTGGGGGAGGCTTAGTGAAGCCTGGAGGGTCCCTGAAACT
CTCCTGTGCAGCCTCTGGATTCACTTTCAGTAGCTTTGGAATGCACTGGG
TTCGTCAGGCTCCAGAGAAGGGGCTGGAGTGGGTCGCATACATTAGTAGT
GACGGTAGTACCATCTACTATGCAGACACAGTGAAGGGCCGATTCACCAT
GTCCAGAGACAATCCCAAGAACACCCTGTTCCTGCAAATGACCAGTCTAA
GGTCTGAGGACACGGCCATGTATTACTGT
EA1.20 reverse

(SEQ ID No 33)

GGTGACTGAGGTTCCTTGACCCCAATAGTCCATAGCATACCCGCCCAGCC
ATCTTACACAGTAATACATGGCCGTGTCCTCAGACCTTAGACTGGTCATT
TGCAGGAACAGGGTGTTCTTGGGATTGTCTCTGGACATGGTGAATCGGCC
CTTCACTGTGTCTGCATAGTAGATGGTACTACCGTCACTACTAATGTATG
CGACCCACTCCAGCCCCTTCTCTGGAGCCTGACGAACCCAGTGCATTCCA
AAGCTACTGAAAGTGAATCCAGAGGCTGCACAGGAGAGTTTCAGGGACCC
TCCAGGCTTCACTAAGCCTCCCCCAGATTCCACCAGCTTCACCTCGGATC
CACCACCACCGGAGCCGCCACCACCACGTTTTATTTCCAGCTTGGTGCCT
CCACCGAACGTCCACGGAAGATCCCTACTGTGCTGACAGTAATAGGTTGC
AGCATCCTCCTCTTCCACAGGATGGATGTTGAGGGTGAAGTCTGTCCCAG
ACCCACTGCCACTGAACCTGGCAGGGACCCCAGATTCTAGGTTGGATGCA
AGATAGATGAGGAGCTTGGGTGGCTGTCCTGGTTTCTGTTGGTACCAGTG
CATATAACTATAGCCAGATGTAGTGACACTTTTGCTGGCCCTGCATGAGA
TGGTGGCCCCTCTGCCCCGGAGACACAAGTAAGGAAGCAGGAGATTGGGT
CATCACAATATCTTTGTAGTCCGCCATGGCCGGCTGGGCCGCGAGTAATA
ACAATCCAGCGGCTGCCGTAGGCAATAGGTATTTCA
EA1.23 forward

(SEQ ID No 34)

TAGATAACGAGGGCAAATCATGAAATACCTATTGCCTACGGCAGCCGCTG
GATTGTTATTACTCGCGGCCCAGCCGGCCATGGCGGACTACAAAGATATT
GTGATGACCCAATCTCCTGCTTCCTTACTTGTGTCTCCGGGGCAGAGGGC
CACCATCTCATGCAGGGCCAGCAAAAGTGTCACTACATCTGGCTATAGTT
ATATGCACTGGTACCAACAGAAACCAGGACAGGCACCCAAGCTCCTCATC
TATCTTGCATCCAACCTAGAATCTGGGGTCCCTGCCAGGTTCAGTGGCAG
TGGGTCTGGGACAGACTTCACCCTCAACATCCATCCTGTGGAAGAGGAGG
ATGCTGCAACCTATTACTGTCAGCAC
EA1.23 reverse

(SEQ ID No 35)

CGAGGAGACGGTGACTGAGGTTCCTTGACCCCAATAGTCCATAGCATACC
CGCCCAGCCATCTTACACAGTAATACATGGCCGTGTCCTCAGACCTTAGA
CTGGTCATTTGCAGGAACAGGGTGTTCTTGGGATTGTCTCTGGACATGGT
GAATCGGCCCTTCACTGTGTCTGCATAGTAGATGGTACTACCGTCACTAC
TAATGTATGCGACCCACTCCAGCCCCTTCTCTGGAGCCTGACGAACCCAG
TGCATTCCAAAGCTACTGAAAGTGAATCCAGAGGCTGCACAGGAGAGTTT
CAGGGACCCTCCAGGCTTCACTAAGCCTCCCCCAGATTCCACCAGCTTCA
CCTCGGATCCACCACCACCGGAGCCGCCACCACCACGTTTTATTTCCAGC
TTGGTGCCTCCACCGAACGTCCACGGAAGATCCCTACTGTGCTGACAGTA
ATAGGTTGCAGCATCCTCCTCTTCCACAGGATGGATGTTGAGGGTGAAGT
CTGTCCCAGACCCACTGCCACTGAACCTGGCAGGGACCCCAGATTCTAGG
TTGGATGCAAGATAGATGAGGAGCTTGGGTGGCTGTCCTGGTTTCTGTTG
GTACCAGTGCATATAACTATAGCCAGATGTAGTGACACTTTTGCTGGCCC
TGCATGAGATGGTGGCCCTCTGCCCCGGAGACACAAGTAAGGAAGCAGGA
GATTGGGTCATCACAATATCTTTGTAGTCCGCCATGGCCGGCTGGGCCGC
GAGTAATAACAATCCAGCGGCTGCCGTAGGCAATAGGTAT
EA10.1 forward

(SEQ ID No 36)

GATTACGAATTTCTAGATAACGAGGGCAAATCATGAAATACCTATTGCCT
ACGGCAGCCGCTGGATTGTTATTACTCGCGGCCCAGCCGGCCATGGCGGA
CTACAAAGATATTCAGATGATACAGTCTCCATCCAGTCTGTCTGCATCCC
TTGGAGACACAATTACCATCACTTGCCATGCCAGTCAGAACATTAATGTT
TGGTTAAGCTGGTACCAGCAGAAACCAGGAAATATTCCTAAACTATTGAT
CTATAAGGCTTCCAACTTGCACACAGGCGTCCCATCAAGGTTTAGTGGCA
GTGGATCTGGAACAGGTTTCACATTAACCATCAGCAGCCTGCAGCCTGAA
GACATTGCCACTTACTACTGTCAACAGGGTCAAAGTTATCCGTGGACGTT
CGGTGGAGGCACCAAGCTGGAAATCAAACGTGGTGGTGGTGGTTCTGGTG
GTGGTGGTTCTGGCGGCGGCGGCTCCGGTGGTGGTGGATCCGAGGTTCAG
CTGCAGCAGTCTGGAGCTGAGCTGATGAAGCCTGGGGCCTCAGTGAAGAT
ATCCTGCATGGCTACTGGCTACACATTCAGTAGCCACTGGATAGAGTGGG
TAAAGCAGAGGCCTGGACATGGCCTTGAGTGGATTGGAGAGATTTTACCT
GGAAGTGGTAGTACTAACTACATGAGAAGTTCAAGGACAAGGCCACATTC
ACTGCAGATACATCCTCCAACACAGCCTACATGCAACTCATCAGCCTGAC
ATCTGAGGAC
EA10.1 reverse

(SEQ ID No 37)

TGAGAGTGGTGCCTTGGCCCCAGTAGTCAAAGGAGTTATTACCGTAGTCC
CGTCTTGCACAGTAATAGACGGCAGAGTCCTCAGATGTCAGGCTGATGAG
TTGCATGTAGGCTGTGTTGGAGGATGTATCTGCAGTGAATGTGGCCTTGT
CCTTGAACTTCTCATTGTAGTTAGTACTACCACTTCCAGGTAAAATCTCT
CCAATCCACTCAAGGCCATGTCCAGGCCTCTGCTTTACCCACTCTATCCA
GTGGCTACTGAATGTGTAGCCAGTAGCCATGCAGGATATCTTCACTGAGG
CC
EA10.4 forward

(SEQ ID No 38)

TATGACCATGATTACGAATTTCTAGATAACGAGGGCAAATCATGAAATAC
CTATTGCCTACGGCAGCCGCTGGATTGTTATTACTCGCGGCCCAGCCGGC
CATGGCGGACTACAAAGATATTGTGATGACCCAATCTCCTGCTTCCTTAC
TTGTGTCTCCGGGGCAGAGGGCCACCATCTCATGCAGGGCCAGCAAAAGT
GTCACTACATCTGGCTATAGTTATATGCACTGGTACCAACAGAAACCAGG
ACAGCCACCCAAGCTCCTCATCTATCTTGCATCCAACCTAGAATCTGGGG
TCCCTGCCAGGTTCAGTGGCAGTGGGTCTGGGACAGACTTCACCCTCAAC
ATCCATCCTGTGGAAGAGGAGGATGCTGCAACCTATTACTGTCAGCACAG
TAGGGATCTTCCGTGGACGTTCGGTGGAGGCACCAAGCTGGAAATAAAAC
GTGGTGGTGGCGGCTCCGGTGGTGGTGGATCCGAGGTGAAGCTGGTGGAA
TCTGGGGGAGGCTTAGTGAAGCCTGGAGGGTCCCTGAAACTCTCCTGTGC
AGCCTCTGGATTCACTTTCAGTAGCTTTGGAATGCACTGGGTTCGTCAGG
CTCCAAAAAA
EA10.4 reverse

(SEQ ID No 39)

GAGGAGACGGTGACTGAGGTTCCTTGACCCCATAGTCCATAGCATACCCG
CCCAGCCATCTTACACAGTAATACATGGCCGTGTCCTCAGACCTTAGACT
GGTCATTTGCAGGAACAGGGTGTTCTTGGGATTGTCTCTGGACATGGTGA
ATCGGCCCTTCACTGTGTCTGCATAGTAGATGGTACTACCGTCACTACTA
ATGTATGCGACCCACTCCAGCCCCTTCTCTGGAGCCTGACGAACCCAGTG
CATTCCAAAGCTACTGAAAGTGAATCCAGAGGCTGCACAGGAGAGTTTCA
GGGACCCTCCAGGCTTCACTAAGCCTCCCCCAGATTCCACCAGCTTCACC
TCGGATCCACCACCACCGGAGCCGCCACCACCACGTTTTATTTCCAGCTT
GGTGCCTCCACCGAACGTCCACGGAAGATCCCTACTGTGCTGACAGTAAT
AGGTTGCAGCATCCTCCTCTTCCACAGGATGGATGTTGAGGGTGAAGTCT
GTCCCAGACCCACTGCCACTGAACCTGGCAGGGACCCCAGATTCTAGGTT
GGATGCAAGATAGATGAGGAGCTTGGGTGGCTGTCCTGGTTTCTGTTGGT
ACCAGTGCATATAACTATAGCCAGATGTAGTGACACTTTTGCTGGCCCTG
CATGAGATGGTGGCCCTCTGCCCCGGAGACACAAGTAAGGAAGCAGGAGA
TTGGGTCATCACAATATCTTTGTAGTCCGCCATGGCCGGCTGGGCCGCGA
GTAATAACAATCCAGCGGCTGCCGTAGGCAATAG

Antibodies can be used to produce mimics of the epitopes that they recognise thus producing a ‘surrogate antigen’, often termed an anti-idiotypic antibody. Without wishing to be bound by theory, it is believed that the complementary determining regions (CDRs) of an antibody are the predominant parts of antibody structure involved in epitope recognition (Reference 32). If an antibody is selected that binds to the original antibody (e.g. an anti-EA1 scFv) the structure of the antibody so selected (in particular the CDRs) mimics that of the original epitope (part of EA1). This method thus utilises antibodies to produce an immune response to a defined epitope of a specific antigen, in the same way as an antigen is used in a vaccine preparation, but the target and the area to which it binds is more defined (Reference 33). Thus if a target of a pathogen, such as EA1, known to be immunogenic in humans, is found to have a role in enhancing protection to anthrax infection, it can be used in vaccine production (References 34, 35).

The therapeutic use of anti-spore antibodies, such as the EA1 single chains could also be used to enhance protection to anthrax infection. This usually occurs as the presence of antibody enhances components of the human immune system or aids in preventing the establishment of infection. This is commonly undertaken through the administration of a humanised form of the single chain as described by Zhou et al., (Reference 31). In both cases the CDRs described herein that bind specifically to the target EA1, would remain the same.

### REFERENCES

1. Borrebaeck, C. A. K., and Ohlin, M (2002). Antibody evolution beyond nature. Nat. Biotechnol. 20, 1189-1190.
2. Cai, X. and Garen, A. (1995). Anti-melanoma antibodies from melanoma patients immunised with genetically autologous tumour cells: Selection of specific antibodies from single-chain Fv fusion phage libraries. Proc. Natl. Acad. Sci. USA. 92, 6537-6541.
3. Emanuel, P. A., Dang, J., Gebhardt, J. S., Aldrich, J., Garber, E. A. E., Kulaga, H., Stopa, P., Valdes, J. J. and Dion-Scultz. (2000). Recombinant antibodies: a new reagent for biological detection. Biosens. Bioelectron. 14, 751-759.
4. Framatico, P. M., Strobaugh, T. M., Medina, M. B., Gehring, A. G. (1998). Detection of Escherichia Coli 0157:h7 using a surface plasmon resonance biosensor. Biotechnol. Tech. 12, 571-576.
5. Glasner, S. M., Yelton, D. E. and Huse, W. D. (1992). Antibody engineering by codon-based mutagenesis in a filamentous phage vector system. J. Immunol. 15, 3903-3913.
6. Helgason, E., Okstad, O. A., Caugant, D. A., Johansen, H. A., Fouet, A., Mock, M. (2000). Bacillus anthracis, Bacillus cereus and B. thuringiensis—one species on the basis of genetic evidence. Appl. Environ. Microbiol. 66, 2627-2630.
7. Holt, L. J., Büssow, K., Walter, G. and Tomlinson, I. M. (2000). By-passing selection: direct screening for antibody-antigen interactions using protein arrays. Nucleic Acids Research. 28 (15), E72-e72.
8. Ivanova, N., Sorokin, A., Anderson, I., Galleron, N., Candelon, B., Kapatral, V., Bhattacharyya, A., Reznik, G., Mikhailova, N., Lapidus, A. et al., (2003). Genome sequence of Bacillus cereus and comparative analysis with B. anthracis. Nature. 423, 87-91.
9. Irving, R. A., Kortt, A. A. and Hudson, P. J. (1996). Affinity maturation of recombinant antibodies using E. coli mutator cells. Immunotechnology. 2, 127-143.
10. Jermatus, L., Honeggar, A., Schwesinger, P., Hanes, J and Plückthun. (2001). Tailoring in vitro evolution for protein affinity or stability. Proc. Natl. Acad. Sci. 98 (1), 75-80.
11. Jung, S., Honegger, A. and Plückthun, A. (1999). Selection for improved protein stability by phage display. J. Mol. Biol. 294, 163-180.
12. Kirkham, P. M., Neri, D and Winter, G. (1999). Towards the design of an antibody that recognises a given protein epitope. J. Mol. Biol. 285, 909-915.
13. Krebber, A., Bornhauser, S., Burmester, Bumester, J., Honegger, A., Willunda, J., Bosshard, H. R., Plückthun, A. (1997) Reliable cloning of functional antibody variable domains from hybridomas and spleen cell repertoires employing a reengineered phage display system. J. Immunol. Methods. 201, 35-55.
14. Krebs, B., Rauchenberger, R., Reiffert, S., Rothe, C., Tesar, M., Thomassen, E., Cao, M., Dreier, T., Fischer, Höβ, A., et al., (2004). High-throughput generation and engineering of recombinant human antibodies. J. Immunol. Methods. 254, 67-84.
15. Labadie, J and Desnier, I. (1992). Selection of cell wall antigens for the rapid detection of bacteria by immunological methods. J. Appl. Bacteriol. 72(3), 220-226.
16. Lindner, P., Bauer, K., Krebber, A., Nieba, L., Kremmer, E., Krebber, C., Honegger, A., Klinger, B., Mockikat, R. and Plückthun, A. (1997). Specific Detection of His-tagged proteins with recombinant anti-His Tag scFv-phosphatase or scFv fusions. Biotechniques. 22, 140-149.
17. McCarthy, B. J. and Hill, A. S. (2001). Altering the fine specificity of an anti-Legionella single chain antibody by a single amino acid insertion. J. Immunol. Methods. 251, 137-147.
18. Mesnage, S., Tosi-Couture, E., Mock, M., Gounon, P. and Fouet, A. (1997). Molecular characterisation of the Bacillus anthracis main S-layer component: evidence that it is the major cell associated antigen. Mol. Microbiol. 23(6), 1147-1155.
19. Miyazaki, C., Iba., Y., Yamada, Y., Takahashi, H., Sawada, J. and Kurosawa, Y. (1999). Changes in the specificity of antibodies by site specific mutagenesis followed by random mutagenesis. Prot. Eng. 12(5), 407-415
20. Mössner, E and Plückthun, A. (2001). Directed evolution with fast efficient selection technologies. Chimia. 55, 324-328.
21. Perkins, E. A. and Squirrel, D. J. (2000). Development of instrumentation to allow the detection of microorganisms using light scattering in combination with surface plasmon resonance. Biosens. Bioelectron. 14, 853-859.
22. Petrenko, V. A. and Vodyanoy, V. J. (2003). Phage display for detection of biological threat agents. J. Microbiol. Methods, 53: 253-262.
23. Piervincenzi, R. T., Reichart, W. M. and Hellinga, H. W. (1998). Genetic engineering of a single-chain antibody fragment for surface immobilisation in an optical biosensor. Biosens. Bioelectron. 13(3 and 4), 305-312.
24. Read, T., Peterson, S. N. Tournasse, N., Baillie, L. W., Paulsen, I. T. Nelson, K. E., Tettelin, H., Fouts, D. E., Eisen, J. A., Gill, S. R., et al. (2003) The genome sequence of Bacillus anthracis Ames and comparison to closely related bacteria. Nature 423, 81-86.
25. Redmond, C., Baillie, L. W. J., Hibbs, S., Moir, A. J. G. and Moir, A. (2004). Identification of proteins in the exosporium of Bacillus anthracis. Microbiology. 150, 355-363.
26. Skerra, A (2000). Engineered protein scaffolds for molecular recognition. J. Mol. Recognit. 13, 167-187.
27. Studentsov, Y. Y. Schiffmann, M., Strickler, H. D., Ho, G. Y. F., Pang, Y. S., Schiler, J., Herrero, R. and Burk, R. D. (2002). Enhanced Enzyme-Linked Immunosorbent assay for detection of antibodies to virus like particles of Human Papillomavirus. J. Clin. Microbiol. 40(5), 1755-1760.
28. Todd, S. J., Moir, A. J., Johnson, M. J., and Moir, A (2003). Genes of Bacillus cereus and Bacillus anthracis encoding proteins of the exosporium. J. Bacteriol. 185(11), 3373-3378.
29. Williams, D. D., Benedeck, O. and Turnbough, C. L. (2003). Species-specific peptide ligands for the detection of Bacillus anthracis spores. Appl. Env. Microbiol. 69(10), 6288-6293.
30. Williams, D. D. and Turnbough, C. L. (2004). Surface layer protein EA1 is not a component of Bacillus anthracis spores but is a persistent contamination in spore preparations. J. Bacteriol. 186(2), 556-569.
31. Zhou, B., Wirsching, P. and Janda, K. D. (2002). Human antibodies against spores of the genus Bacillus: A model study for detection of and protection against anthrax and the bioterrorist threat. Proc. Natl. Acad. Sci. 99(8), 5421-5246.
32. Goldbaum, F. A., Velikovsky, A., Dall'Acqua, W., Fossati, C. A., Fields, B. A., Braden, B. C., Poljak, R. J. and Mariuzza, R. A (1997). Characterisation of anti-idiotypic antibodies that bind antigen and an anti-idiotype. Proc. Natl. Acad, Sci. USA. 94; 8697-8701
33. Vogel., M., Miescher, S., Kuhn, S., Zurcher, A. W., Stadler, M. B., Ruf, C., Effenberger, F., Kricek, F. and Stadler, B. M. (2000). Mimicry of human IgE epitopes by anti-idiotypic antibodies. J. Mol. Biol. 293; 729-735.
34. Baillie, L., Hebdon, R., Flick-Smith, H. and Williamson, D. (2003). Characterisation of the immune response to the UK human anthrax vaccine. FEMS Immunology and Medical Microbiology 36; 83-86.
35. Ariel, N., Zvi, A., Makarova, K. S., Chitlaru, T., Elhanny, E., Velan, B., Cohen, S., Friedlander, A. M. and Shafferman, A. (2003). Genome-based bioinformatic selection of chromosomal Bacillus anthracis putative vaccine candidates coupled with proteomic identification of surface associated antigens. Infection and Immunity. 71 (8); 4563-4579.
36. Martin, A. C. R. (2001). Protein sequence & structure analysis of antibody variable domains. In: Antibody Engineering Lab Manual. eds Kontermann, R. & Dubel, S., Springer-Verlag, Heidelberg. pp 423-439.

1. An antibody which binds to anthrax with high specificity.

2. An antibody that binds specifically to a Bacillus anthracis protein EA1 without cross reactivity with other Bacillus species.

3. The antibody of claim 2 having an amino acid sequence comprising at least one amino acid sequence selected from the group consisting of
SEQ ID No. 17;
SEQ ID No. 10;
SEQ ID No. 18;
SEQ ID No. 19;
SEQ ID No. 4;
SEQ ID No. 13;
SEQ ID No. 20;
SEQ ID No. 14 and
SEQ ID No. 21 or a variant thereof.

4. The antibody of claim 2 wherein the antibody contains at least one hypervariable region selected from the group consisting of CDR-L1, CDR-L2, CDR-L3, CDR-H1, CDR-H2 and CDR-H3 and wherein:
CDR-L1 comprises SEQ ID No 17 or SEQ ID No. 10;
CDR-L2 comprises SEQ ID No. 18;
CDR-L3 comprises SEQ ID No. 19;
CDR-H1 comprises SEQ ID No. 4 or SEQ ID No. 13;
CDR-H2 comprises SEQ ID No. 20 or SEQ ID No. 14; and
CDR-H3 comprises SEQ ID No. 21.

5. The antibody of claim 2 wherein the antibody has an amino acid sequence comprising any one of SEQ ID NOS 1-16 or a variant thereof or a fragment thereof.

6. The antibody of claim 5 wherein the antibody has an amino acid sequence comprising:
SEQ ID NOS 1, 2, 3, 4, 5 and 6;
SEQ ID NOS 7, 2, 3, 4, 8 and 6;
SEQ ID NOS 7, 2, 3, 4, 5 and 6;
SEQ ID NOS 9, 2, 3, 4, 5 and 6;
SEQ ID NOS 10, 11, 12, 13, 14 and 15 or,
SEQ ID NOS 7, 2, 3, 4, 5 and 16

7. A method of detecting anthrax comprising binding an antibody to anthrax spores, wherein the antibody binds with high specificity to Bacillus anthracis without cross reactivity with other Bacillus species.

8. A nucleic acid molecule encoding the antibody of claim 1.

9. The nucleic acid molecule of claim 8, wherein the nucleic acid molecule has a nucleic acid sequence comprising any of SEQ ID NOS 28-39 or a variant thereof.

10. A pharmaceutical composition comprising the antibody of claim 1.

11. The composition of claim 10, wherein said composition is a vaccine.

12-13. (canceled)

14. The composition of claim 11 wherein the vaccine comprises an anti-idiotypic antibody to the antibody of claim 1.

15. An anti-idiotypic antibody to the antibody of claim 1.

16. A method of selecting an antibody from an antibody library comprising simultaneously contacting the library with a plurality of potentially cross-reacting antibody targets.


SEQUENCE LISTING (40 sequences)

SEQ ID NO: 1 - protein, 15 aa, Mus musculus
AASKSVTTSGYSYMH

SEQ ID NO: 2 - protein, 7 aa, Mus musculus
LASNLES

SEQ ID NO: 3 - protein, 9 aa, Mus musculus
QHSRDLPWT

SEQ ID NO: 4 - protein, 5 aa, Mus musculus
SFGMH

SEQ ID NO: 5 - protein, 15 aa, Mus musculus
YISSDGSTIYYADTV

SEQ ID NO: 6 - protein, 9 aa, Mus musculus
WLGGYAMDY

SEQ ID NO: 7 - protein, 15 aa, Mus musculus
RASKSVTTSGYSYMH

SEQ ID NO: 8 - protein, 15 aa, Mus musculus
YISSDLSTIYYADTV

SEQ ID NO: 9 - protein, 14 aa, Mus musculus
ASKSVTTSGYSYMH

SEQ ID NO: 10 - protein, 11 aa, Mus musculus
HASQNINVWLS

SEQ ID NO: 11 - protein, 7 aa, Mus musculus
KASNLHT

SEQ ID NO: 12 - protein, 9 aa, Mus musculus
QQGQSYPWT

SEQ ID NO: 13 - protein, 5 aa, Mus musculus
SHWIE

SEQ ID NO: 14 - protein, 17 aa, Mus musculus
EILPGSGSTNYNEKFKD

SEQ ID NO: 15 - protein, 10 aa, Mus musculus
RDYGNNSFDY

SEQ ID NO: 16 - protein, 17 aa, Mus musculus
WLGGYAMDYKEPQSPSP

SEQ ID NO: 17 - protein, 14 aa, Mus musculus
ASKSVTTSGYSYMH

SEQ ID NO: 18 - protein, 3 aa, Mus musculus
ASN

SEQ ID NO: 19 - protein, 8 aa, Mus musculus
  MISC_FEATURE (2)..(5): Xaa is an amino acid
QXXXXPWT

SEQ ID NO: 20 - protein, 15 aa, Mus musculus
  MISC_FEATURE (6)..(6): Xaa is an amino acid
YISSDXSTIYYADTV

SEQ ID NO: 21 - protein, 9 aa, Mus musculus
  MISC_FEATURE (1)..(3): Xaa is an amino acid
XXXGXXXDY

SEQ ID NO: 22 - protein, 240 aa, Mus musculus
DYKDIVMTQSPASLLVSPGQRATISCAASKSVTTSGYSYMHWYQQKPGQPPKLLIYLASNLESGVPARFSGSGSGTDFTLNIHPVEEEDAATYYCQHSRDLPWTFGGGTKLEIKRGGGGSGGGGSEVKLVESGGGLVKPGGSLKLSCAASGFTFSSFGMHWVRQAPEKGLEWVAYISSDGSTIYYADTVKGRFTMSRDNPKNTLFLQMTSLRSEDTAMYYCVRWLGGYAMDYWGQGTSVT

SEQ ID NO: 23 - protein, 240 aa, Mus musculus
DYKDIVMTQSPASLLVSPGQRATISCRASKSVTTSGYSYMHWYQQKPGQPPKLLIYLASNLESGVPARFSGSGSGTDFTLNIHPVEEEDAATYYCQHSRDLPWTFGGGTKLEIKRGGGGSGGGGSEVKLVESGGGLVKPGGSLKLSCAASGFTFSSFGMHWVRQAPEKGLEWVAYISSDLSTIYYADTVKGRFTMSRDNPKNTLFLQMTSLRSEDTAMYYCVRWLGGYAMDYWGQGTSVT

SEQ ID NO: 24 - protein, 240 aa, Mus musculus
DYKDIVMTQSPASLLVSPGQRATISCRASKSVTTSGYSYMHWYQQKPGQPPKLLIYLASNLESGVPARFSGSGSGTDFTLNIHPVEEEDAATYYCQHSRDLPWTFGGLTKLEIKRGGGGSGGGGSEVKLVESGGGLVKPGGSLKLSCAASGFTFSSFGMHWVRQAPEKGLEWVAYISSDGSTIYYADTVKGRFTMSRDNPKNTLFLQMTSLRSEDTAMYYCVRWLGGYAMDYWGQGTSVT

SEQ ID NO: 25 - protein, 242 aa, Mus musculus
DYKDIVMTQSPASLLVSPGQRATISCASKSVTTSGYSYMHWYQQKPGQPPKLLIYLASNLESGVPARFSGSGSGTDFTLNIHPVEEEDAATYYCQHSRDLPWTFGGGTKLEIKRGGGGSGGGGSEVKLVESGGGLVKPGGSLKLSCAASGFTFSSFGMHWVRQAPEKGLEWVAYISSDGSTIYYADTVKGRFTMSRDNPKNTLFLQMTSLRSEDTAMYYCVRWLGGYAMDYWGQGTSVTVSS

SEQ ID NO: 26 - protein, 246 aa, Mus musculus
DYKDIQMIQSPSSLSASLGDTITITCHASQNINVWLSWYQQKPGNIPKLLIYKASNLHTGVPSRFSGSGSGTGFTLTISSLQPEDIATYYCQQGQSYPWTFGGGTKLEIKRGGGGSGGGGSGGGGSGGGGSEVQLQQSGAELMKPGASVKISCMATGYTFSSHWIEWVKQRPGHGLEWIGEILPGSGSTNYNEKFKDKATFTADTSSNTAYMQLISLTSEDSAVYYCARRDYGNNSFDYWGQGTTL

SEQ ID NO: 27 - protein, 242 aa, Mus musculus
DYKDIVMTQSPASLLVSPGQRATISCRASKSVTTSGYSYMHWYQQKPGQPPKLLIYLASNLESGVPARFSGSGSGTDFTLNIHPVEEEDAATYYCQHSRDLPWTFGGGTKLEIKRGGGGSGGGGSEVKLVESGGGLVKPGGSLKLSCAASGFTFSSFGMHWVRQAPEKGLEWVAYISSDGSTIYYADTVKGRFTMSRDNPKNTLFLQMTSLRSEDTAMYYCVRWLGGYAMDYGVKEPQSPSP

SEQ ID NO: 28 - DNA, 778 bp, Mus musculus
tatgaccatgattacgaatttctagataacgagggcaaatcatgaaatacctattgcctacggcagccgctggattgttattactcgcggcccagccggccatggcggactacaaagatattgtgatgacccaatctcctgcttccttacttgtgtctccggggcagagggccaccatctcatgcagggccagcaaaagtgtcactacatctggctatagttatatgcactggtaccaacagaaaccaggacagccacccaagctcctcatctatcttgcatccaacctagaatctggggtccctgccaggttcagtggcagtgggtctgggacagacttcaccctcaacatccatcctgtggaagaggaggatgctgcaacctattactgtcagcacagtagggatcttccgtggacgttcggtggaggcaccaagctggaaataaaacgtggtggtggcggctccggtggtggtggatccgaggtgaagctggtggaatctgggggaggcttagtgaagcctggagggtccctgaaactctcctgtgcagcctctggattcactttcagtagctttggaatgcactgggttcgtcaggctccagagaaggggctggagtgggtcgcatacattagtagtgacggtagtaccatctactatgcagacacagtgaaaggccgattcaccatgtccagagacaatcccaagaacaccctgttcctgcaaatgaccagtctaaggtctgaagacacggccatgtattactgtgtaagatg

SEQ ID NO: 29 - DNA, 720 bp, Mus musculus
gtaatacatggccgtgtcctcagaccttagactggtcatttgcaggaacagggtgttcttgggattgtctctggacatggtgaatcggcccttcactgtgtctgcatagtagatggtactaccgtcactactaatgtatgcgacccactccagccccttctctggagcctgacgaacccagtgcattccaaagctactgaaagtgaatccagaggctgcacaggagagtttcagggaccctccaggcttcactaagcctcccccagattccaccagcttcacctcggatccaccaccaccggagccgccaccaccacgttttatttccagcttggtgcctccaccgaacgtccacggaagatccctactgtgctgacagtaataggttgcagcatcctcctcttccacaggatggatgttgagggtgaagtctgtcccagacccactgccactgaacctggcagggaccccagattctaggttggatgcaagatagatgaggagcttgggtggctgtcctggtttctgttggtaccagtgcatataactatagccagatgtagtgacacttttgctggccctgcatgagatggtggccctctgccccggagacacaagtaaggaagcaggagattgggtcatcacaatatctttgtagtccgccatggccggctgggccgcgagtaataacaatccagcggctgccgtaagcaataggta

SEQ ID NO: 30 - DNA, 779 bp, Mus musculus
gaaacagctatgaccatgattacgaatttctagataacgagggcaaatcatgaaatacctattgcctacggcagccgctggattgttattactcgcggcccagccggccatggcggactacaaagatattgtgatgacccaatctcctgcttccttacttgtgtctccggggcagagggccaccatctcatgcagggccagcaaaagtgtcactacatctggctatagttatatgcactggtaccaacagaaaccaggacagccacccaagctcctcatctatcttgcatccaacctagaatctggggtccctgccaggttcagtggcagtgggtctgggacagacttcaccctcaacatccatcctgtggaagaggaggatgctgcaacctattactgtcagcacagtagggatcttccgtggacgttcggtggaggcaccaagctggaaataaaacgtggtggtggcggctccggtggtggtggatccgaggtgaagctggtggaatctgggggaggcttagtgaagcctggagggtccctgaaactctcctgtgcagcctctggattcactttcagtagctttggaatgcactgggttcgtcaggctccagagaaggggctggagtgggtcgcatacattagtagtgacggtagtaccatctactatgcagacacagtgaagggccgattcaccatgtccagagacaatcccaagaacaccctgttcctgcaaatgaccagtctaaggtctgaggacacggccatgtattactgtg

SEQ ID NO: 31 - DNA, 302 bp, Mus musculus
tgagagtggtgccttggccccagtagtcaaaggagttattaccgtagtcccgtcttgcacagtaatagacggcagagtcctcagatgtcaggctgatgagttgcatgtaggctgtgttggaggatgtatctgcagtgaatgtggccttgtccttgaacttctcattgtagttagtactaccacttccaggtaaaatctctccaatccactcaaggccatgtccaggcctctgctttacccactctatccagtggctactgaatgtgtagccagtagccatgcaggatatcttcactgaggcc

SEQ ID NO: 32 - DNA, 779 bp, Mus musculus
ggaaacagctatgaccatgattacgaatttctagataacgagggcaaatcatgaaatacctattgcctacggcagccgctggattgttattactcgcggcccagccggccatggcggactacaaagatattgtgatgacccaatctcctgcttccttacttgtgtctccggggcagagggccaccatctcatgcagggccagcaaaagtgtcactacatctggctatagttatatgcactggtaccaacagaaaccaggacagccacccaagctcctcatctatcttgcatccaacctagaatctggggtccctgccaggttcagtggcagtgggtctgggacagacttcaccctcaacatccatcctgtggaagaggaggatgctgcaacctattactgtcagcacagtagggatcttccgtggacgttcggtggaggcaccaagctggaaataaaacgtggtggtggcggctccggtggtggtggatccgaggtgaagctggtggaatctgggggaggcttagtgaagcctggagggtccctgaaactctcctgtgcagcctctggattcactttcagtagctttggaatgcactgggttcgtcaggctccagagaaggggctggagtgggtcgcatacattagtagtgacggtagtaccatctactatgcagacacagtgaagggccgattcaccatgtccagagacaatcccaagaacaccctgttcctgcaaatgaccagtctaaggtctgaggacacggccatgtattactgt

SEQ ID NO: 33 - DNA, 786 bp, Mus musculus
ggtgactgaggttccttgaccccaatagtccatagcatacccgcccagccatcttacacagtaatacatggccgtgtcctcagaccttagactggtcatttgcaggaacagggtgttcttgggattgtctctggacatggtgaatcggcccttcactgtgtctgcatagtagatggtactaccgtcactactaatgtatgcgacccactccagccccttctctggagcctgacgaacccagtgcattccaaagctactgaaagtgaatccagaggctgcacaggagagtttcagggaccctccaggcttcactaagcctcccccagattccaccagcttcacctcggatccaccaccaccggagccgccaccaccacgttttatttccagcttggtgcctccaccgaacgtccacggaagatccctactgtgctgacagtaataggttgcagcatcctcctcttccacaggatggatgttgagggtgaagtctgtcccagacccactgccactgaacctggcagggaccccagattctaggttggatgcaagatagatgaggagcttgggtggctgtcctggtttctgttggtaccagtgcatataactatagccagatgtagtgacacttttgctggccctgcatgagatggtggcccctctgccccggagacacaagtaaggaagcaggagattgggtcatcacaatatctttgtagtccgccatggccggctgggccgcgagtaataacaatccagcggctgccgtaggcaataggtatttca

SEQ ID NO: 34 - DNA, 376 bp, Mus musculus
tagataacgagggcaaatcatgaaatacctattgcctacggcagccgctggattgttattactcgcggcccagccggccatggcggactacaaagatattgtgatgacccaatctcctgcttccttacttgtgtctccggggcagagggccaccatctcatgcagggccagcaaaagtgtcactacatctggctatagttatatgcactggtaccaacagaaaccaggacagccacccaagctcctcatctatcttgcatccaacctagaatctggggtccctgccaggttcagtggcagtgggtctgggacagacttcaccctcaacatccatcctgtggaagaggaggatgctgcaacctattactgtcagcac

SEQ ID NO: 35 - DNA, 790 bp, Mus musculus
cgaggagacggtgactgaggttccttgaccccaatagtccatagcatacccgcccagccatcttacacagtaatacatggccgtgtcctcagaccttagactggtcatttgcaggaacagggtgttcttgggattgtctctggacatggtgaatcggcccttcactgtgtctgcatagtagatggtactaccgtcactactaatgtatgcgacccactccagccccttctctggagcctgacgaacccagtgcattccaaagctactgaaagtgaatccagaggctgcacaggagagtttcagggaccctccaggcttcactaagcctcccccagattccaccagcttcacctcggatccaccaccaccggagccgccaccaccacgttttatttccagcttggtgcctccaccgaacgtccacggaagatccctactgtgctgacagtaataggttgcagcatcctcctcttccacaggatggatgttgagggtgaagtctgtcccagacccactgccactgaacctggcagggaccccagattctaggttggatgcaagatagatgaggagcttgggtggctgtcctggtttctgttggtaccagtgcatataactatagccagatgtagtgacacttttgctggccctgcatgagatggtggccctctgccccggagacacaagtaaggaagcaggagattgggtcatcacaatatctttgtagtccgccatggccggctgggccgcgagtaataacaatccagcggctgccgtaggcaataggtat

SEQ ID NO: 36 - DNA, 760 bp, Mus musculus
gattacgaatttctagataacgagggcaaatcatgaaatacctattgcctacggcagccgctggattgttattactcgcggcccagccggccatggcggactacaaagatattcagatgatacagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatccgtggacgttcggtggaggcaccaagctggaaatcaaacgtggtggtggtggttctggtggtggtggttctggcggcggcggctccggtggtggtggatccgaggttcagctgcagcagtctggagctgagctgatgaagcctggggcctcagtgaagatatcctgcatggctactggctacacattcagtagccactggatagagtgggtaaagcagaggcctggacatggccttgagtggattggagagattttacctggaagtggtagtactaactacatgagaagttcaaggacaaggccacattcactgcagatacatcctccaacacagcctacatgcaactcatcagcctgacatctgaggac

SEQ ID NO: 37 - DNA, 302 bp, Mus musculus; identical to SEQ ID NO: 31

SEQ ID NO: 38 - DNA, 610 bp, Mus musculus
tatgaccatgattacgaatttctagataacgagggcaaatcatgaaatacctattgcctacggcagccgctggattgttattactcgcggcccagccggccatggcggactacaaagatattgtgatgacccaatctcctgcttccttacttgtgtctccggggcagagggccaccatctcatgcagggccagcaaaagtgtcactacatctggctatagttatatgcactggtaccaacagaaaccaggacagccacccaagctcctcatctatcttgcatccaacctagaatctggggtccctgccaggttcagtggcagtgggtctgggacagacttcaccctcaacatccatcctgtggaagaggaggatgctgcaacctattactgtcagcacagtagggatcttccgtggacgttcggtggaggcaccaagctggaaataaaacgtggtggtggcggctccggtggtggtggatccgaggtgaagctggtggaatctgggggaggcttagtgaagcctggagggtccctgaaactctcctgtgcagcctctggattcactttcagtagctttggaatgcactgggttcgtcaggctccaaaaaa

SEQ ID NO: 39 - DNA, 784 bp, Mus musculus
gaggagacggtgactgaggttccttgaccccatagtccatagcatacccgcccagccatcttacacagtaatacatggccgtgtcctcagaccttagactggtcatttgcaggaacagggtgttcttgggattgtctctggacatggtgaatcggcccttcactgtgtctgcatagtagatggtactaccgtcactactaatgtatgcgacccactccagccccttctctggagcctgacgaacccagtgcattccaaagctactgaaagtgaatccagaggctgcacaggagagtttcagggaccctccaggcttcactaagcctcccccagattccaccagcttcacctcggatccaccaccaccggagccgccaccaccacgttttatttccagcttggtgcctccaccgaacgtccacggaagatccctactgtgctgacagtaataggttgcagcatcctcctcttccacaggatggatgttgagggtgaagtctgtcccagacccactgccactgaacctggcagggaccccagattctaggttggatgcaagatagatgaggagcttgggtggctgtcctggtttctgttggtaccagtgcatataactatagccagatgtagtgacacttttgctggccctgcatgagatggtggccctctgccccggagacacaagtaaggaagcaggagattgggtcatcacaatatctttgtagtccgccatggccggctgggccgcgagtaataacaatccagcggctgccgtaggcaatag

SEQ ID NO: 40 - protein, 5 aa, Artificial sequence
  Synthetic sequence Linker sequence
GGGGS